(19) United States
(12) Patent Application Publication
(10) Pub. No.: US 2022/0378337 A1
(43) Pub. Date: Dec. 1, 2022

(54) Adaptive Systems for Continuous Glucose Monitoring

(71) Applicant: Dexcom, Inc., San Diego, CA (US)

(72) Inventors: Stephen Vanslyke, Carlsbad, CA (US); Arturo Garcia, La Mesa, CA (US); Andrew Parker, San Diego, CA (US); Peter Simpson, Cardiff, CA (US); Leif Bowman, San Diego, CA (US); David Price, Carlsbad, CA (US); Richard Kelley, San Diego, CA (US); Zebediah McDaniel, San Diego, CA (US); Andrew Pal, San Diego, CA (US); Nicholas Polytaridis, San Diego, CA (US); Sumi Mikami, San Diego, CA (US); Apurv Kamath, San Diego, CA (US); Lauren Jepson, San Diego, CA (US)

(73) Assignee: Dexcom, Inc., San Diego, CA (US)

(21) Appl. No.: 17/746,743

(22) Filed: May 17, 2022

Related U.S. Application Data
(60) Provisional application No. 63/189,460, filed on May 17, 2021.

(51) Int. Cl. A61B 5/145; G16H 40/67; A61B 5/00; G16H 20/60

(52) U.S. Cl. CPC A61B 5/14532; G16H 40/67; A61B 5/746; A61B 5/743; G16H 20/60

(57) ABSTRACT

In implementations of adaptive systems for continuous glucose monitoring (CGM), a computing device implements an adaptive system to receive glucose data describing user glucose values measured by a sensor of a CGM system, the sensor is inserted at an insertion site. The adaptive system accesses orientation data describing forces measured by an accelerometer of the CGM system, and the adaptive system identifies a location of the insertion site based on the orientation data. Modified glucose data is generated by modifying the user glucose values based on the location of the insertion site. The adaptive system generates an indication of the modified glucose data for display in a user interface of a display device.

### RELATED APPLICATION

This application claims the benefit of U.S. Provisional Patent Application No. 63/189,460, filed May 17, 2021, and titled “Adaptive Systems for Continuous Glucose Monitoring,” the entire disclosure of which is hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events, such as eating or exercising.

Advances in medical technologies have facilitated development of various systems for monitoring blood glucose levels, including continuous glucose monitoring (CGM) systems, which measure and record glucose concentrations in substantially real-time. A user of a CGM system inserts a glucose sensor subcutaneously at an insertion site (e.g., on the user's abdomen, arm, or buttock) and the user wears the glucose sensor for a period of time which can be several days or longer. The CGM system interfaces with a computing device and the computing device receives data from a transmitter of the CGM system describing measured glucose concentrations at the insertion site.

While the glucose sensor is inserted, the user of the CGM system (or another user such as a physician or a parent) can interact with a user interface of the computing device to view the glucose concentrations measured by the glucose sensor. After wearing the glucose sensor for the period of time, the user replaces the sensor with a new glucose sensor which the user wears for another period of time. By design, this replacement causes the CGM system to be modified (e.g., to operate using a different glucose sensor) and/or one or more aspects of its deployment to be modified (e.g., to operate at a different location). However, conventional CGM systems are not capable of identifying or quantifying effects of such modifications on the glucose concentrations measured and communicated to the computing device for viewing. This is a shortcoming of conventional CGM systems especially in scenarios where the modifications significantly impact or adversely affect performance of the system, e.g., a new sensor is defective.

### SUMMARY

In order to overcome the limitations of conventional systems, techniques and systems are described for adaptive continuous glucose monitoring (CGM). In an example, glucose data is received describing user glucose values measured by a glucose sensor of a CGM system. For example, the glucose sensor is inserted at an insertion site by a user of the CGM system to measure glucose values of the user.

The CGM system may also include an accelerometer, which measures forces and generates orientation data describing the measured forces. For instance, forces caused by movements of the user of the CGM system while the glucose sensor is inserted at the insertion site may be measured by the accelerometer. A location of the insertion site is determined based on characteristics and/or patterns of those forces as described by the orientation data.

An adaptive system is implemented to generate modified glucose data by modifying the user glucose values based on the location of the insertion site. In one example, the glucose data includes an error component such as an incorrect user glucose value because of the location of the insertion site, e.g., the location is not an intended location for inserting the glucose sensor and causes erroneous glucose values to be produced. By modifying the glucose values, though, the modified glucose data does not include the error component. For example, the modified glucose data does not include the incorrect user glucose value. An indication of the modified glucose data is generated for display in a user interface via a display device.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ techniques described herein.

FIG. 2 depicts an example of the continuous glucose monitoring (CGM) system of FIG. 1 in greater detail.

FIG. 3 depicts an example implementation in which a computing device communicates orientation data to a storage device of a virtual container and an adaptive system accesses non-glucose data stored in the virtual container in association with generating modified data.

FIG. 4 depicts an example implementation of the adaptive system of FIG. 3 in greater detail.

FIG. 5 illustrates a representation of session data describing historic user glucose values measured by a single-use glucose sensor since the single-use glucose sensor was installed in a continuous glucose monitoring (CGM) system.

FIG. 6 illustrates a representation of modified session data usable to generate a glucose value report.

FIG. 7 illustrates a representation of a glucose value report displayed in a user interface of a computing device.

FIG. 8 illustrates a representation of glucose data and modified glucose data.

FIG. 9 illustrates a representation of a user interface for confirming a determined location of a glucose sensor insertion site.

FIG. 10 illustrates a representation of a user interface for identifying which meal of multiple purchased meals was consumed by a user of a continuous glucose monitoring (CGM) system.

FIG. 11 illustrates a representation of a user interface for decision support in meal planning.

FIG. 12 illustrates a representation of a user interface for setting up a continuous glucose monitoring (CGM) system.

FIG. 13 illustrates a representation of a user interface for testing alarms of a continuous glucose monitoring (CGM) system.

FIG. 14 is a flow diagram depicting a procedure in an example implementation in which glucose data describing user glucose values is received, modified glucose data is generated based on a location of an insertion site of a glucose sensor, and an indication of the modified glucose data is generated for display in a user interface.

FIG. 15 is a flow diagram depicting a procedure in an example implementation in which glucose data describing user glucose values is received, modified glucose data is generated based an anomaly of an insertion site of a glucose sensor, and an indication of the modified glucose data is generated for display in a user interface.

FIG. 16 is a flow diagram depicting a procedure in an example implementation in which glucose data describing user glucose values is received, a modification amount is determined based on non-glucose data, and modified glucose data is generated by modifying the user glucose values based on the modification amount.

FIG. 17 is a flow diagram depicting a procedure in an example implementation in which session data describing historic user glucose values is received, modified session data is generated by removing historic user glucose values from the session data that were measured by a glucose sensor during a temporal window, and a glucose value report is generated based on the modified session data.

FIG. 18 is a flow diagram depicting a procedure in an example implementation in which glucose data describing user glucose values is received, a modification amount is determined based on non-glucose data describing historic perspiration values of a user of the CGM system, and modified glucose is generated by modifying the user glucose values based on the modification amount.

FIG. 19 is a flow diagram depicting a procedure in an example implementation in which glucose data describing user glucose values is received, a glucose value event is predicted, and modified glucose data is generated because the glucose value event did not occur.

FIG. 20 is a flow diagram depicting a procedure in an example implementation in which glucose data describing user glucose values is received, a location of an insertion site of a glucose sensor is identified, and an indication of an error component included in the glucose data is generated for display in a user interface based on the location of the insertion site.

FIG. 21 illustrates an example system that includes an example computing device that is representative of one or more computing systems and/or devices that may implement the various techniques described herein.

### DETAILED DESCRIPTION

Overview

A continuous glucose monitoring (CGM) system measures glucose concentrations via a sensor which is inserted subcutaneously and worn by a user of the CGM system for a period of time indicated by the sensor. After this period of time, the sensor is replaced with a new sensor which is a modification to the CGM system. The different location where the new sensor is worn by the user is also a modification to the CGM system. These modifications are normally minor but can sometimes significantly impact operation of the system, for example, if the new sensor is defective or damaged. Conventional CGM systems are not capable of identifying and quantifying an impact of these modifications or adapting based on a quantified impact. In order to overcome the limitations of conventional systems, techniques and systems are described for adaptive continuous glucose monitoring.

In accordance with the described techniques, glucose data is received describing user glucose values measured by a glucose sensor of a CGM system. The glucose sensor is inserted at an insertion site by a user of the CGM system, and a computing device receives the glucose data from the glucose sensor via a transmitter of the CGM system. Once the glucose data is received, a user may interact with a user interface of the computing device to view the user glucose values described by the glucose data.

An adaptive system of the CGM system receives or accesses orientation data generated by an accelerometer of the CGM system. This orientation data describes forces measured by the accelerometer due to movements of the user while the glucose sensor is inserted at the insertion site. The adaptive system determines a location of the insertion site based on the forces measured by the accelerometer as described by the orientation data. In one example, the location is determined by comparing the measured forces with a characteristic force pattern associated with the location.

The adaptive system can also determine that a location of the insertion site is not an intended location for inserting the glucose sensor, such as when the location of the insertion site is not on the user's abdomen, arm, or buttock. In scenarios where it is determined that the location of the insertion site is not an intended location for inserting the glucose sensor, the glucose data may include an error component, e.g., causing an incorrect user glucose value.

The adaptive system generates modified glucose data by modifying the user glucose values based on the location of the insertion site. In accordance with the described techniques, the adaptive system modifies the glucose values so that the modified glucose data does not include the error component (e.g., the incorrect user glucose value) which was included in the glucose data. An indication of the modified glucose data is generated for display in the user interface of the computing device. By generating the modified glucose data so that it does not include the error component caused by the location of the insertion site, the described systems improve CGM technology relative to conventional systems which are not capable of determining the location of the insertion site and modifying the user's glucose values according to the determined location. Additionally, this modification causes the described systems to present values that more accurately reflect the user's glucose level than the values presented by conventional techniques, which fail to correct glucose values based on insertion site location.

In the following description, an example environment is first described that is configured to employ the techniques described herein. Example implementation details and procedures are then described which may be performed in the example environment as well as other environments. Performance of the example procedures is not limited to the example environment and the example environment is not limited to performance of the example procedures.

Example Environment

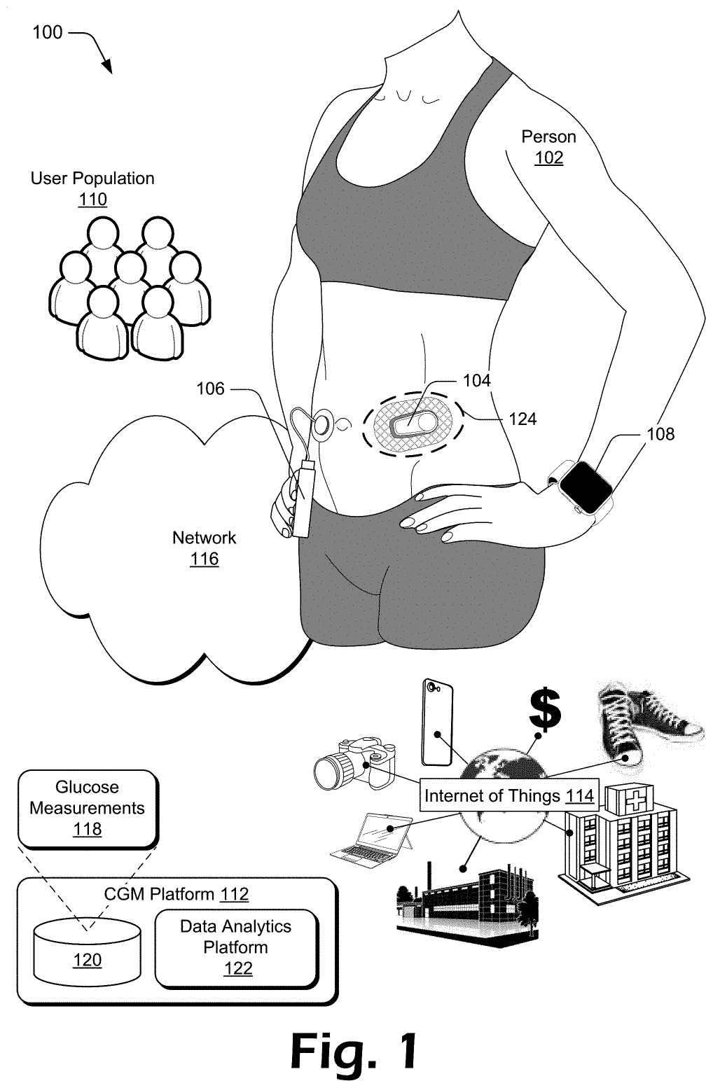

FIG. 1 is an illustration of an environment 100 in an example implementation that is operable to employ techniques described herein. The illustrated environment 100 includes person 102 (e.g., a user), who is depicted wearing a continuous glucose monitoring (CGM) system 104, an insulin delivery system 106, and a computing device 108. The illustrated environment 100 also includes other users in a user population 110, a CGM platform 112, and an Internet of Things 114 (IoT 114). The CGM system 104, insulin delivery system 106, computing device 108, user population 110, CGM platform 112, and IoT 114 are communicatively coupled, including via a network 116.

Alternatively or additionally, one or more of the CGM system 104, the insulin delivery system 106, or the computing device 108 are communicatively coupled in other ways, such as using one or more wireless communication protocols and/or techniques. By way of example, the CGM system 104, the insulin delivery system 106, and the computing device 108 are configured to communicate with one another using one or more of Bluetooth (e.g., Bluetooth Low Energy links), near-field communication (NFC), 5G, and so forth. In some examples, the CGM system 104, the insulin delivery system 106 and/or the computing device 108 are capable of radio frequency (RF) communications and include an RF transmitter and an RF receiver. In these examples, one or more RFIDs are usable for identification and/or tracking of the CGM system 104, the insulin delivery system 106, and/or the computing device 108 within the environment 100. For example, the CGM system 104, the insulin delivery system 106, and the computing device 108 are configured to leverage various types of communication to form a closed-loop system between one another.

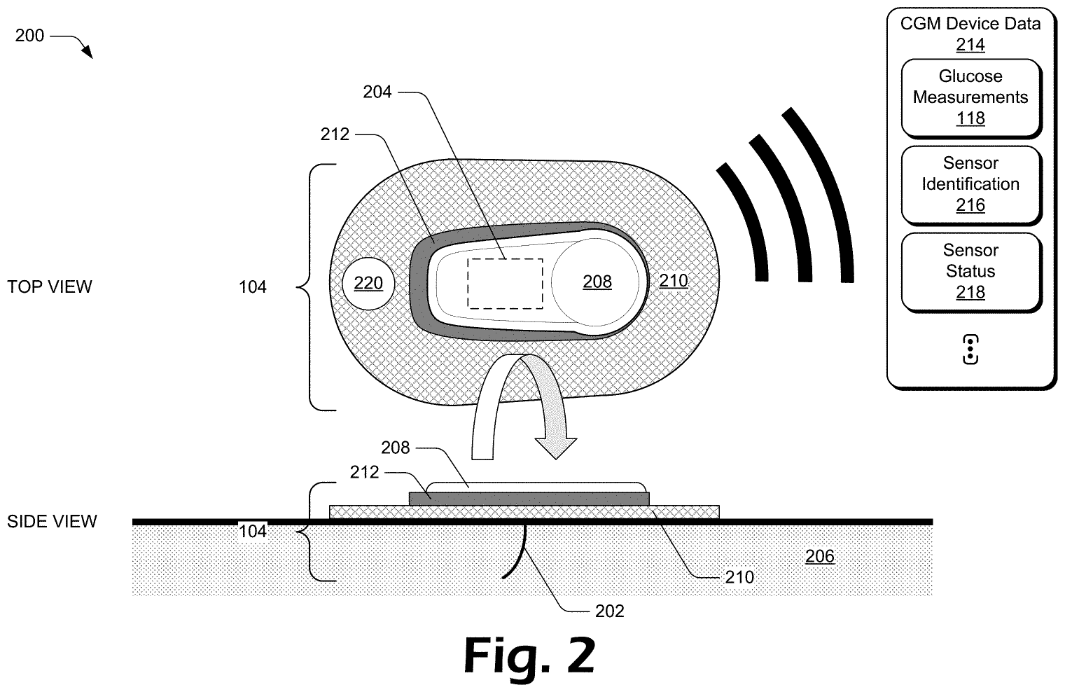

In accordance with the described techniques, the CGM system 104 is configured to continuously monitor glucose levels of the person 102. For example, in some implementations the CGM system 104 is configured with a CGM sensor that continuously detects analytes indicative of the person's 102 glucose level and enables generation of glucose measurements. In the illustrated environment 100, these measurements are represented as glucose measurements 118. This functionality and further aspects of the CGM system's 104 configuration are described in further detail below with respect to FIG. 2.

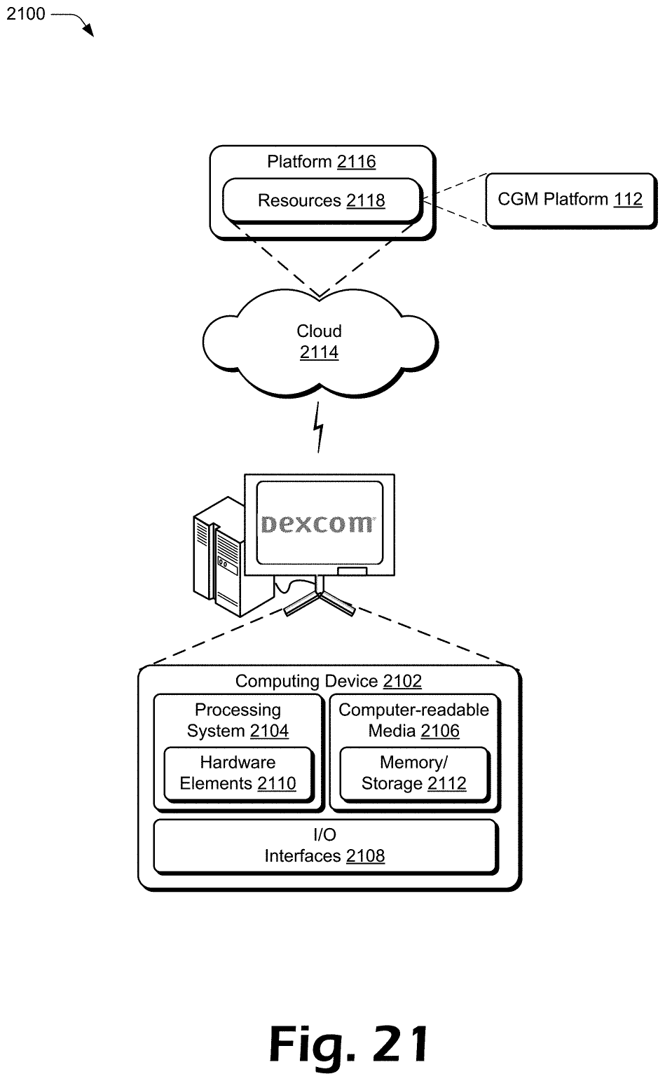

In one or more implementations, the CGM system 104 transmits the glucose measurements 118 to the computing device 108, via one or more of the communication protocols described herein, such as via wireless communication. The CGM system 104 is configured to communicate these measurements in real-time (e.g., as the glucose measurements 118 are produced) using a CGM sensor. Alternatively or additionally, the CGM system 104 is configured to communicate the glucose measurements 118 to the computing device 108 at designated intervals (e.g., every 30 seconds, every minute, every five minutes, every hour, every six hours, every day, and so forth). In some implementations, the CGM system 104 is configured to communicate glucose measurements responsive to a request from the computing device 108 (e.g., a request initiated when the computing device 108 generates glucose measurement predictions for the person 102, a request initiated when displaying a user interface conveying information about the person's 102 glucose measurements, combinations thereof, and so forth). Accordingly, the computing device 108 is configured to maintain the glucose measurements 118 of the person 102 at least temporarily (e.g., by storing glucose measurements 118 in computer-readable storage media, as described in further detail below with respect to FIG. 21).

Although illustrated as a wearable device (e.g., a smart watch), the computing device 108 is implementable in a variety of configurations without departing from the spirit or scope of the described techniques. By way of example and not limitation, in some implementations the computing device 108 is configured as a different type of mobile device (e.g., a mobile phone or tablet device). In other implementations, the computing device 108 is configured as a dedicated device associated with the CGM platform 112 (e.g., a device supporting functionality to obtain the glucose measurements 118 from the CGM system 104, perform various computations in relation to the glucose measurements 118, display information related to the glucose measurements 118 and the CGM platform 112, communicate the glucose measurements 118 to the CGM platform 112, combinations thereof, and so forth). In some examples in which the computing device 108 is configured as a mobile phone, the computing device 108 excludes functionality otherwise available via mobile phone configurations when implemented in a dedicated CGM device configuration, such as functionality to make phone calls, capture images, utilize social networking applications, and the like. In other examples in which the computing device is configured as a mobile phone, the computing device 108 does not exclude functionality otherwise available via mobile phone configurations when implemented in the dedicated CGM device configuration.

In some implementations, the computing device 108 is representative of more than one device. For instance, the computing device 108 is representative of both a wearable device (e.g., a smart watch) and a mobile phone. In such multiple device implementations, different ones of the multiple devices are capable of performing at least some of the same operations, such as receiving the glucose measurements 118 from the CGM system 104, communicating the glucose measurements 118 to the CGM platform 112 via the network 116, displaying information related to the glucose measurements 118, and so forth. Alternatively or additionally, different devices in the multiple device implementations support different capabilities relative to one another, such as capabilities that are limited by computing instructions to specific devices.

In some example implementations where the computing device 108 represents separate devices, (e.g., a smart watch and a mobile phone) one device is configured with various sensors and functionality to measure a variety of physiological markers (e.g., perspiration, heart rate, heart rate variability, breathing, rate of blood flow, and so on) and activities (e.g., steps, elevation changes, eating, drinking, exercising, and the like) of the person 102. Continuing this example multiple device implementation, another device is not configured with such sensors or functionality, or includes a limited amount of such sensors or functionality. For instance, one of the multiple devices includes capabilities not supported by another one of the multiple devices, such as a camera to capture images of meals useable to predict future glucose levels, an amount of computing resources (e.g., battery life, processing speed, etc.) that enables a device to efficiently perform computations in relation to the glucose measurements 118. Even in scenarios where one of the multiple devices (e.g., a smartphone) is capable of carrying out such computations, computing instructions may limit performance of those computations to one of the multiple devices, so as not to burden multiple devices with redundant computations, and to more efficiently utilize available resources. In this manner, the computing device 108 is representative of a variety of different configurations and representative of different numbers of devices beyond the specific example implementations described herein.

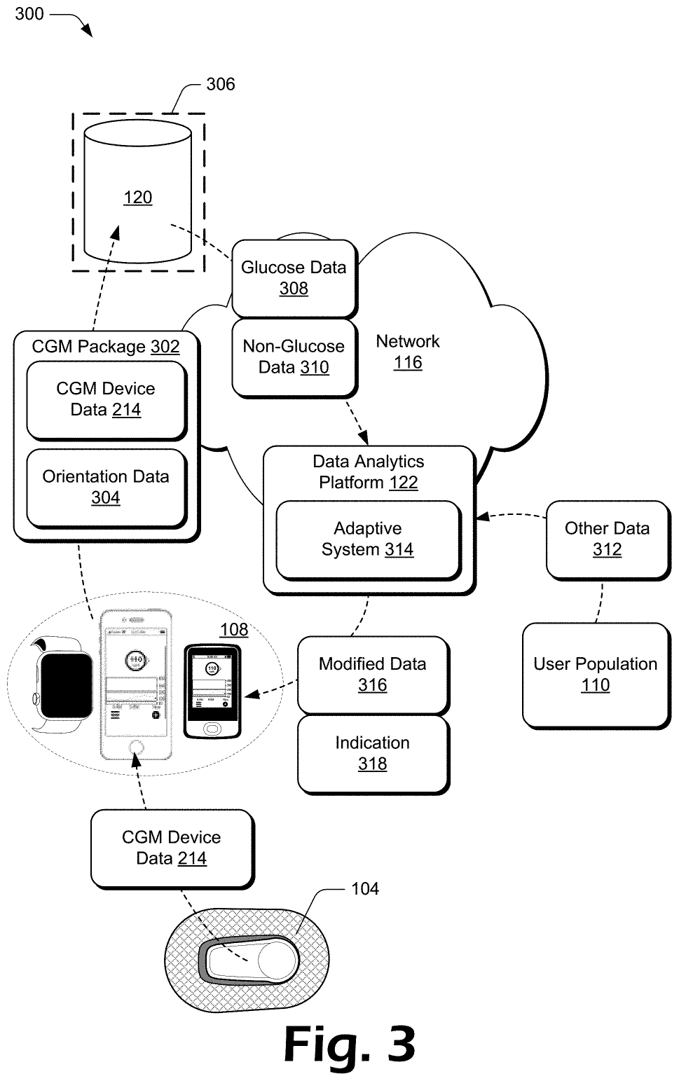

As mentioned above, the computing device 108 communicates the glucose measurements 118 to the CGM platform 112. In the illustrated environment 100, the glucose measurements 118 are depicted as being stored in storage device 120 of the CGM platform 112. In some examples, the storage device 120 includes or is included in a virtual container which limits access to data stored in the storage device 120 as described in greater detail with respect to FIG. 3. The storage device 120 is representative of one or more types of storage (e.g., databases) capable of storing the glucose measurements 118. In this manner, the storage device 120 is configured to store a variety of other data in addition to the glucose measurements 118.

For instance, in accordance with one or more implementations, the person 102 represents a user of at least the CGM platform 112 and one or more other services (e.g., services offered by one or more third party service providers). For example, the person 102 is able to be associated with personally attributable information (e.g., a username) and may be required, at some time, to provide authentication information (e.g., password, biometric data, telemedicine service information, and so forth) to access the CGM platform 112 using the personally attributable information. The storage device 120 is configured to maintain this personally attributable information, authentication information, and other information pertaining to the person 102 (e.g., demographic information, healthcare provider information, payment information, prescription information, health indicators, user preferences, account information associated with a wearable device, social network account information, other service provider information, and the like).

The storage device 120 is further configured to maintain data pertaining to other users in the user population 110. As such, the glucose measurements 118 in the storage device 120 are representative of both the glucose measurements from a CGM sensor of the CGM system 104 worn by the person 102 as well as glucose measurements from CGM sensors of CGM systems worn by other persons represented in the user population 110. In a similar manner, the glucose measurements 118 of these other persons of the user population 110 may be communicated by respective devices via the network 116 to the CGM platform 112, such that other persons are associated with respective user profiles in the CGM platform 112.

The data analytics platform 122 represents functionality to process the glucose measurements 118-alone and/or along with other data maintained in the storage device 120. Based on this processing, the CGM platform 112 is configured to provide notifications in relation to the glucose measurements 118 (e.g., alerts, alarms, recommendations, or other information generated based on the processing). For instance, the CGM platform 112 is configured to provide notifications to the person 102, to a medical service provider associated with the person 102, combinations thereof, and so forth. Although depicted as separate from the computing device 108, portions or an entirety of the data analytics platform 122 are alternatively or additionally configured for implementation at the computing device 108. The data analytics platform 122 is further configured to process additional data obtained via the IoT 114.

To supply some of this additional information beyond previous glucose measurements, the IoT 114 is representative of various sources capable of providing data that describes the person 102 and the person's 102 activity as a user of one or more service providers and activity with the real world. By way of example, the IoT 114 includes various devices of the user (e.g., cameras, mobile phones, laptops, exercise equipment, and so forth). In this manner, the IoT 114 is configured to provide information about interactions of the user with various devices (e.g., interaction with web-based applications, photos taken, communications with other users, and so forth). Alternatively or additionally, the IoT 114 may include various real-world articles (e.g., shoes, clothing, sporting equipment, appliances, automobiles, etc.) configured with sensors to provide information describing behavior, such as steps taken, force of a foot striking the ground, length of stride, temperature of a user (and other physiological measurements), temperature of a user's surroundings, types of food stored in a refrigerator, types of food removed from a refrigerator, driving habits, and so forth.

Alternatively or additionally, the IoT 114 includes third parties to the CGM platform 112, such as medical providers (e.g., a medical provider of the person 102) and manufacturers (e.g., a manufacturer of the CGM system 104, the insulin delivery system 106, or the computing device 108) capable of providing medical and manufacturing data, respectively, to platforms that track the person's 102 exercise and nutrition intake that can be leveraged by the data analytics platform 122. Thus, the IoT 114 is representative of devices and sensors capable of providing a wealth of data without departing from the spirit or scope of the described techniques.

As described in greater detail with respect to FIG. 2, the person 102 attaches the CGM system 104 to the person's 102 body such that a glucose sensor of the CGM system 104 is inserted at an insertion site (e.g., below the person's 102 skin). The glucose sensor insertion site is intended to be located in an indicated location (e.g., the person's 102 abdomen or buttocks). In some scenarios in which the glucose sensor insertion site is not located in an indicated location, glucose measurements 118 taken by the CGM system 104 may be inaccurate. In these scenarios, the CGM system 104 is capable of determining when the glucose sensor insertion site is not located in an indicated location. In response to such a determination, the CGM system 104 adapts to correct potential inaccuracies in the glucose measurements 118.

Consider examples in which the CGM system 104 includes at least one accelerometer that measures forces from movements (e.g., acceleration) of the person 102 while the glucose sensor of the CGM system 104 is inserted at an insertion site of the person 102. In some of these examples, the CGM system 104 includes a piezoelectric accelerometer, a piezoresistive accelerometer, a capacitive accelerometer, and so forth. In other examples, the CGM system 104 includes an accelerometer implemented using micro-electrical mechanical systems (MEMS).

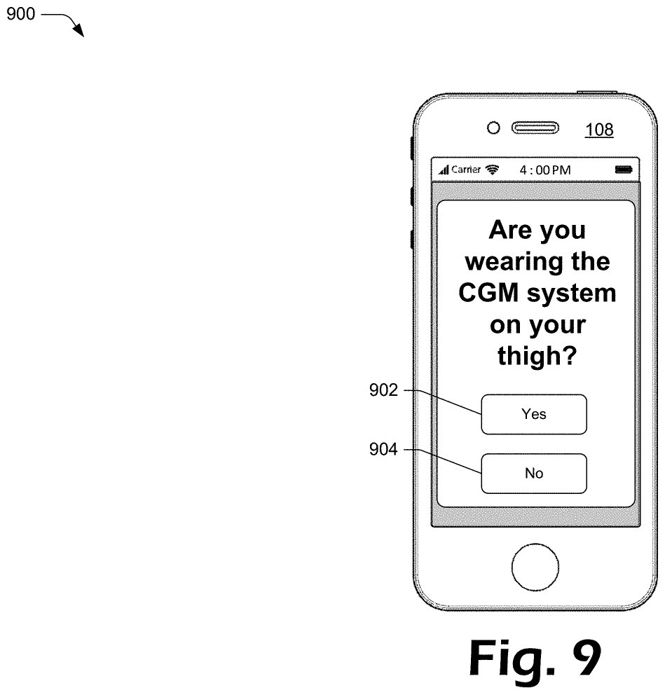

The CGM system 104 communicates data describing forces measured by the accelerometer to the computing device 108 and the computing device 108 processes this data to determine a location 124 of the glucose sensor insertion site. To do so in one example, the computing device 108 compares the forces measured by the accelerometer with multiple characteristic force patterns that are each associated with a particular insertion site location on the person 102. In this example, the computing device 108 identifies the location 124 based on this comparison. As shown, the location 124 is on an abdomen of the person 102 which is an indicated location of the glucose sensor insertion site. An example in which the location 124 is not an indicated location and the CGM system 104 corrects glucose measurements 118 taken from the non-indicated location is described in greater detail with respect to FIG. 9.

Consider an example in which the CGM system 104 includes a photodiode sensor that measures reflected light which may be transmitted by a light emitting diode of the CGM system 104. In this example, the photodiode sensor is disposed in close proximity to the glucose sensor insertion site (e.g., the location 124) such that light data describing reflected light measured by the photodiode sensor can be processed to determine an anomaly of the insertion site. For example, the anomaly of the insertion site is a tattoo, a scar tissue, a skin irritation, and so forth.

In examples in which the location 124 is not the abdomen or a buttock of the person 102 and/or there is an anomaly of the insertion site of the glucose sensor, data describing glucose measurements 118 of the person 102 taken by the CGM system 104 can include an error component. The error component is an error related to at least one of the glucose measurements 118 such as the at least one glucose measurement 118 has a value that is too high, too low, undeterminable, etc. The computing device 108 (e.g., and/or the CGM system 104) is capable of leveraging a variety of different types of data from various sensors and/or input devices to process the data describing glucose measurement 118 of the person 102 and generate modified glucose measurement data which does not include the error component.

In some examples, the CGM system 104 and/or the computing device 108 includes a heart rate monitor such as an optical heart rate monitor capable of measuring the person's 102 heart rate, heart rate variability, oxygen saturation, etc. In one example, the computing device 108 receives heart rate data (e.g., describing the person's 102 heart rate and/or heart rate variability) from an electronic heart rate monitor. In another example, the computing device 108 receives the heart rate data from the CGM system 104. The heart rate data is useable to predict changes in the person's 102 glucose levels, confirm an accuracy of the glucose measurements 118, and so forth.

For example, the CGM system 104 and/or the computing device 108 includes a perspiration sensor which detects increases and decreases in the person's 102 perspiration. In some examples, the perspiration sensor detects the person's 102 perspiration by detecting increases and decreases in analytes associated with perspiration. Examples of analytes associated with perspiration include urea, uric acid, ionic potassium, ionic sodium, ionic chloride, etc.

In a few examples, the perspiration sensor is configured to detect analytes having a significance to the person's 102 glucose regulation such as glycated hemoglobin and/or ketones. For example, the computing device 108 receives perspiration data describing increases and decreases in the person's 102 perspiration from the perspiration sensor. In another example, the computing device 108 receives the perspiration data from the CGM system 104. The computing device 108 processes the perspiration data to recommend actions which the person 102 can perform to increase the person's 102 time in range in one example.

In an example, the computing device 108 includes an image capture device such as a camera and the computing device 108 uses the image capture device to capture images of the person 102. For example, the computing device 108 uses the image capture device to capture images depicting the person's 102 face. The computing device 108 is capable of processing these captured images of the person 102 to determine the person's 102 mood and/or a level of stress that the person 102 is experiencing. For example, the computing device 108 includes a machine learning model trained on training data to generate indications of the person's 102 mood and/or stress level based on an input image depicting the person's 102 face. As used herein, the term “machine learning model” refers to a computer representation that is tunable (e.g., trainable) based on inputs to approximate unknown functions. By way of example, the term “machine learning model” includes a model that utilizes algorithms to learn from, and make predictions on, known data by analyzing the known data to learn to generate outputs that reflect patterns and attributes of the known data. According to various implementations, such a machine learning model uses supervised learning, semi-supervised learning, unsupervised learning, reinforcement learning, and/or transfer learning. For example, the machine learning model is capable of including, but is not limited to, clustering, decision trees, support vector machines, linear regression, logistic regression, Bayesian networks, random forest learning, dimensionality reduction algorithms, boosting algorithms, artificial neural networks (e.g., fully-connected neural networks, deep convolutional neural networks, or recurrent neural networks), deep learning, etc.

By way of example, a machine learning model makes high-level abstractions in data by generating data-driven predictions or decisions from the known input data. In one example, the machine learning model is trained on training data describing images of the person 102. In another example, the machine learning model is trained on training data describing images of the user population 110. The computing device 108 generates mood and/or stress data by processing captured images of the person's 102 face. For example, the computing device 108 is also capable of limiting use of the captured images of the person 102 to determining the person's 102 mood and/or stress level. For example, after processing an image of the person 102 to determine the person's 102 mood and/or stress level, the computing device 108 deletes the image of the person 102.

In various examples, the mood data, the stress data, the perspiration data, the heart rate data, the light data, and/or the location 124 are leverageable to augment the glucose measurements 118 and guide the person's 102 decision making process as part of managing type I or type II diabetes. Specific examples in which the mood data, the stress data, the perspiration data, the heart rate data, and/or the location 124 are used to provide both clinical and lifestyle insights to the person 102 are described in greater detail with respect to FIGS. 9-13. Although examples are described with respect to the glucose measurements 118, it is to be appreciated that glucose is one example analyte and the described systems and techniques are usable with respect to other analytes and/or other analyte monitoring devices. In the context of measuring glucose, e.g., continuously, and obtaining data describing such measurements, consider the following description of FIG. 2.

FIG. 2 depicts an example implementation 200 of the CGM system 104 of FIG. 1 in greater detail. In particular, the illustrated example 200 includes a top view and a corresponding side view of the CGM system 104. The CGM system 104 is illustrated as including a sensor 202 and a sensor module 204. In the illustrated example 200, the sensor 202 is depicted in the side view as inserted subcutaneously into skin 206 (e.g., skin of the person 102). The sensor module 204 is depicted in the top view as a rectangle having a dashed outline. The CGM system 104 is further illustrated as including a transmitter 208. Use of the dashed outline of the rectangle representing sensor module 204 indicates that the sensor module 204 may be housed in, or otherwise implemented within a housing of, the transmitter 208. In this example 200, the CGM system 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206, such as via the attachment mechanism 212. Additionally or alternatively, the transmitter 208 may be incorporated as part of the application assembly, such that the sensor 202, the adhesive pad 210, the attachment mechanism 212, and the transmitter 208 (with the sensor module 204) can all be applied to the skin 206 simultaneously. In one or more implementations, the application assembly is applied to the skin 206 using a separate applicator (not shown). This application assembly may also be removed by peeling the adhesive pad 210 off of the skin 206. In this manner, the CGM system 104 and its various components as illustrated in FIG. 2 represent one example form factor, and the CGM system 104 and its components may have different form factors without departing from the spirit or scope of the described techniques. In some examples, the sensor 202 is a single-use glucose sensor of the CGM system 104. In other examples, the sensor 202 is a reusable glucose sensor of the CGM system 104.

In operation, the sensor 202 is communicatively coupled to the sensor module 204 via at least one communication channel, which can be a “wireless” connection or a “wired” connection. Communications from the sensor 202 to the sensor module 204, or from the sensor module 204 to the sensor 202, can be implemented actively or passively and may be continuous (e.g., analog) or discrete (e.g., digital). The sensor 202 may be a device, a molecule, and/or a chemical that changes, or causes a change, in response to an event that is at least partially independent of the sensor 202. The sensor module 204 is implemented to receive indications of changes to the sensor 202, or caused by the sensor 202. For example, the sensor 202 can include glucose oxidase, which reacts with glucose and oxygen to form hydrogen peroxide that is electrochemically detectable by an electrode of the sensor module 204. In this example, the sensor 202 may be configured as, or include, a glucose sensor configured to detect analytes in blood or interstitial fluid that are indicative of glucose levels using one or more measurement techniques.

In another example, the sensor 202 (or an additional, not depicted, sensor of the CGM system 104) can include first and second electrical conductors and the sensor module 204 can electrically detect changes in electric potential across the first and second electrical conductors of the sensor 202. In this example, the sensor module 204 and the sensor 202 are configured as a thermocouple, such that the changes in electric potential correspond to temperature changes. In some examples, the sensor module 204 and the sensor 202 are configured to detect a single analyte (e.g., glucose). In other examples, the sensor module 204 and the sensor 202 are configured to detect multiple analytes (e.g., sodium, potassium, carbon dioxide, and glucose). Alternatively or additionally, the CGM system 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, although not depicted in the illustrated example of FIG. 2, the sensor module 204 may include a processor and memory. By leveraging such a processor, the sensor module 204 may generate the glucose measurements 118 based on the communications with the sensor 202 that are indicative of one or more changes (e.g., analyte changes, environmental condition changes, and so forth). Based on communications with the sensor 202, the sensor module 204 is further configured to generate CGM device data 214. CGM device data 214 is representative of a communicable package of data that includes at least one glucose measurement 118. Alternatively or additionally, the CGM device data 214 includes other data, such as multiple glucose measurements 118, sensor identification 216, sensor status 218, combinations thereof, and so forth. In one or more implementations, the CGM device data 214 may include other information, such as one or more of temperatures that correspond to the glucose measurements 118 and measurements of other analytes. In this manner, the CGM device data 214 may include various data in addition to at least one glucose measurement 118, without departing from the spirit or scope of the described techniques.

### Additional Sensors

As shown in FIG. 2, the CGM system 104 includes additional sensors 220 which are illustrated relative to the adhesive pad 210 but which may be included in any component of the CGM system 104. For example, the additional sensors 220 can also be independent of and separate from the CGM system 104. In some examples, the additional sensors 220 include a single additional sensor and in other examples the additional sensors 220 represent multiple additional sensors. The additional sensors 220 are communicatively coupled to the sensor module 204 via at least one communication channel. Communications from the additional sensors 220 to the sensor module 204, or from the sensor module 204 to the additional sensors 220 are active or passive, continuous or discrete, wired or wireless, etc. In various examples, sensors included in the additional sensors 220 are at least partially disposed subcutaneously in or under the skin 206, are at least partially disposed in contact with the skin 206 (e.g., a surface of the skin 206), are not in physical contact with a portion of the person 102, and so forth.

In one example, an accelerometer is included in the additional sensors 220 and the accelerometer measures forces from movements of the person 102. The sensor module 204 receives communications from the accelerometer describing measured forces. For example, the sensor module 204 includes force data describing forces measured by the accelerometer as part of the CGM device data 214. In some examples, the sensor module 204 processes the force data to determine a location of the sensor's 202 insertion site. In other examples, the sensor module 204 processes the force data to generate step data describing steps taken by the person 102 and the sensor module 204 includes the step data as part of the CGM device data 214.

Consider an example in which a photodiode sensor is included in the additional sensors 220, and the photodiode sensor measures reflected light transmitted by a light emitting diode (LED) of the additional sensors 220. The additional sensors 220 can include arrays of photodiode sensors and LEDs and/or other light sources, and the sensor module 204 includes processing and memory resources for a processor (e.g., a microprocessor) of the sensor module 204 to control transmission of photons via the LEDs or other light sources and convert (e.g., via the photodiode sensor) reflected photons into electrons. In this manner, patterns in electrical signals corresponding to the electrons are usable to identify an anomaly of the sensor's 202 insertion site.

In some examples, a heart rate monitor is included in the additional sensors 220 which measures the person's 102 heart rate and the person's 102 heart rate variability. In an example in which the heart rate monitor is electrical, the sensor module 204 receives communications from the heart rate monitor describing changes in electric potential corresponding to beats of the person's 102 heart. In this example, the sensor module 204 includes heart rate data describing the changes in electric potential as part of the CGM device data 214. In an example in which the heart rate monitor is optical, the sensor module 204 receives communications from the heart rate monitor describing changes in blood volume corresponding to beats of the person's 102 heart. In this example, the sensor module 204 includes heart rate data describing the changes in blood volume within the CGM device data 214. In one example, the heart rate monitor leverages the photodiode sensor and the LEDs to measure the changes in the person's 102 blood volume.

For example, a perspiration sensor is included in the additional sensors 220 which measures increases and decreases in the person's 102 perspiration. In this example, the sensor module 204 receives communications from the perspiration sensor describing increases and decreases in measured analyte concentrations, and the sensor module 204 includes perspiration data describing the increases and decreases in measured analyte concentrations as part of the CGM device data 214. This perspiration data is usable to infer an amount of stress the person 102 is experiencing, determine that the person 102 is engaging in a physical activity, and so forth.

In operation, the transmitter 208 may transmit the CGM device data 214 wirelessly as a stream of data to the computing device 108. Alternatively or additionally, the sensor module 204 may buffer the CGM device data 214 (e.g., in memory of the sensor module 204) and cause the transmitter 208 to transmit the buffered CGM device data 214 at various intervals, e.g., time intervals (every second, every thirty seconds, every minute, every five minutes, every hour, and so on), storage intervals (when the buffered CGM device data 214 reaches a threshold amount of data or a number of instances of CGM device data 214), combinations thereof, and so forth.

In addition to generating the CGM device data 214 and causing it to be communicated to the computing device 108, the sensor module 204 is configured to perform additional functionality in accordance with one or more implementations. This additional functionality of the sensor module 204 may also include calibrating the sensor 202 initially or on an ongoing basis as well as calibrating any other sensors of the CGM system 104 such as the additional sensors 220. This computational ability of the sensor module 204 is particularly advantageous where connectivity to services via the network 116 is limited or non-existent.

With respect to the CGM device data 214, the sensor identification 216 represents information that uniquely identifies the sensor 202 from other sensors (e.g., other sensors of other CGM systems 104, other sensors implanted previously or subsequently in the skin 206, sensors included in the additional sensors 220, and the like). By uniquely identifying the sensor 202, the sensor identification 216 may also be used to identify other aspects about the sensor 202, such as a manufacturing lot of the sensor 202, packaging details of the sensor 202, shipping details of the sensor 202, and the like. In this way, various issues detected for sensors manufactured, packaged, and/or shipped in a similar manner as the sensor 202 may be identified and used in different ways (e.g., to calibrate the glucose measurements 118, to notify users to change or dispose of defective sensors, to notify manufacturing facilities of machining issues, etc.).

The sensor status 218 represents a state of the sensor 202 at a given time (e.g., a state of the sensor at a same time as one of the glucose measurements 118 is produced). To this end, the sensor status 218 may include an entry for each of the glucose measurements 118, such that there is a one-to-one relationship between the glucose measurements 118 and statuses captured in the sensor status 218 information. Generally, the sensor status 218 describes an operational state of the sensor 202. In one or more implementations, the sensor module 204 may identify one of a number of predetermined operational states for a given glucose measurement 118. The identified operational state may be based on the communications from the sensor 202 and/or characteristics of those communications.

By way of example, the sensor module 204 may include (e.g., in memory or other storage) a lookup table having the predetermined number of operational states and bases for selecting one state from another. For instance, the predetermined states may include a “normal” operation state where the basis for selecting this state may be that the communications from the sensor 202 fall within thresholds indicative of normal operation (e.g., within a threshold of an expected time, within a threshold of expected signal strength, when an environmental temperature is within a threshold of suitable temperatures to continue operation as expected, combinations thereof, and so forth). The predetermined states may also include operational states that indicate one or more characteristics of the sensor's 202 communications are outside of normal activity and may result in potential errors in the glucose measurements 118.

For example, bases for these non-normal operational states may include receiving the communications from the sensor 202 outside of a threshold expected time, detecting a signal strength of the sensor 202 outside a threshold of expected signal strength, detecting an environmental temperature outside of suitable temperatures to continue operation as expected, detecting that the person 102 has changed orientation relative to the CGM system 104 (e.g., rolled over in bed), and so forth. The sensor status 218 may indicate a variety of aspects about the sensor 202 and the CGM system 104 without departing from the spirit or scope of the techniques described herein.

Having considered an example environment and example CGM system, consider now a description of some example details of adaptive systems for continuous glucose monitoring in accordance with one or more implementations.

Adaptive Systems for Continuous Glucose Monitoring

FIG. 3 depicts an example 300 implementation in which a computing device communicates continuous glucose monitoring (CGM) device data to a storage device and an adaptive system receives glucose data and non-glucose data.

The illustrated example 300 includes the CGM system 104 and examples of the computing device 108 introduced with respect to FIG. 1. The illustrated example 300 also includes the data analytics platform 122 and the storage device 120, which, as described above, stores the glucose measurements 118. In the example 300, the CGM system 104 is depicted as transmitting the CGM device data 214 to the computing device 108. As described with respect to FIG. 2, the CGM device data 214 includes the glucose measurements 118 along with other data. The CGM system 104 is configured to transmit the CGM device data 214 to the computing device 108 in a variety of ways.

The illustrated example 300 also includes a CGM package 302. The CGM package 302 is representative of data including the CGM device data 214 (e.g., the glucose measurements 118, the sensor identification 216, and the sensor status 218), orientation data 304, and/or portions thereof. The orientation data 304 describes forces measured by an accelerometer of the CGM system 104. As shown, the CGM package 302 (which includes the orientation data 304) is stored in the storage device 120 and is available to the data analytics platform 122 subject to a virtual container 306 which limits access to data stored in the storage device 120.

### Virtual Container

For example, the virtual container 306 limits access to the orientation data 304 based on a risk classification associated with access to the orientation data 304. In one example, the risk classification for accessing particular data within the virtual container 306 may be based on a risk classification for a medical device which generated the particular data. In this example, the risk classification can be low, moderate, or high based on a corresponding medical device classification. In an example in which multiple medical devices are involved in generating the particular data, the risk classification is assigned based on a highest risk classification for a medical device included in the multiple medical devices.

Consider an example in which the virtual container 306 facilitates access to data included in the CGM package 302 by third-parties (e.g., third-party application developers) by imposing limitations and conditions of the access to the data included in the CGM package 302. In this example, the virtual container 306 imposes use limitations on the data included in the CGM package 302 in order to comply with federal and state regulations. In one example, the virtual container 306 allows the third-parties to access a version of the data included in the CGM package 302 which has been processed to remove all data which is usable to identify the person 102.

For example, third-parties access the version of the data and process the version of the data to gain insights into the version of the data without exposing an identify of the person 102. In some examples, the virtual container 306 is a data store optimized for fast writes and/or API-based access. In other examples, the virtual container 306 co-locates the CGM device data 214 and the orientation data 304 in a secure and privacy compliant manner.

As illustrated in FIG. 3, the data analytics platform 122 is permitted access to data stored in the storage device 120 by the virtual container 306. Accordingly, the data analytics platform 122 is illustrated as having, receiving, and/or transmitting glucose data 308 and non-glucose data 310. In one example, the glucose data 308 describes user glucose values measured by the sensor 202. In this example, the glucose data 308 describes a sequence of glucose measurements 118 from the person 102.

In some examples, the data analytics platform 122 also receives other data 312 which is illustrated as describing the user population 110. For example, the other data 312 describes sequences of glucose measurements 118 from the user population 110. The other data 312 can include data of various types from various sources. Similarly, the non-glucose data 310 includes a variety of different types of data from a variety of different data sources. As shown, the data analytics platform 122 receives the glucose data 308, the non-glucose data 310, and/or the other data 312 and implements an adaptive system 314 to process the glucose data 308, the non-glucose data 310, and/or the other data 312 to generate modified data 316.

### Glucose Sensor Insertion Site

Consider an example in which the glucose data 308 describes a sequence of user glucose values which correspond to glucose measurements 118 from the person 102 wearing the CGM system 104. In this example, the non-glucose data 310 includes the orientation data 304 which describes forces measured by an accelerometer of the CGM system 104. In an example in which the adaptive system 314 includes processor and memory resources, the adaptive system 314 processes the non-glucose data 310 to determine a location of the sensor's 202 insertion site on the person 102. In another example, the adaptive system 314 causes the computing device 108 to process the non-glucose data 310 to determine the location of the sensor's 202 insertion site.

To do so in one example, the adaptive system 314 causes the computing device 108 to compares forces measured by the accelerometer described by the orientation data 304 to characteristic force patterns. In one example, the other data 312 describes the characteristic force patterns. For example, each of these characteristic force patterns is associated with insertion site location for the sensor 202 and the adaptive system 314 causes the computing device 108 to determine a particular insertion site location of the sensor 202 based on a similarity between the forces described by the orientation data 304 and a characteristic force pattern associated with the particular insertion site location. This particular insertion site location corresponds to a location on the person 102 such as the location 124.

Continuing the previous example, the adaptive system 314 (and/or the computing device 108) leverages the particular insertion site location of the sensor 202 to generate modified data 316 by modifying the glucose data 308. For example, the particular insertion site location on the person 102 is not an intended or recommended location for the person 102 to insert the sensor 202 and wear the CGM system 104. As a result of this, the adaptive system 314 (and/or the computing device 108) determines that glucose measurements 118 generated by the CGM system 104 should be increased or decreased to offset inaccuracies in the glucose data 308 resulting from the person 102 inserting the sensor 202 in the particular insertion site location. The adaptive system 314 generates the modified data 316 as describing corrected user glucose values. For example, the modified data 316 describes user glucose values that would have been measured by the sensor 202 if the sensor 202 was inserted at a recommended insertion site location such as the person's 102 abdomen instead of the particular insertion site location.

The adaptive system 314 also generates an indication 318 (e.g., of the modified data 316) for display in a user interface of the computing device 108. In one example, the indication 318 indicates how the glucose data 308 was modified to generate the modified data 316. In another example, the indication 318 is a prompt requesting confirmation that the sensor 202 is inserted at the particular insertion site location. In an additional example, the indication 318 is an alert or an alarm based on the modified data 316. In another example, the indication 318 indicates one or more other locations for inserting the sensor 202 in a next CGM session when the sensor 202 is replaced. In these examples, the data analytics platform 122 implements the adaptive system 314 to generate the modified data 316 based on the orientation data 304.

Consider an example in which the modified data 316 is generated based on an anomaly of the sensor's 202 insertion site. In this example, the CGM system 104 generates light data describing reflected light measured by a photodiode sensor. For example, the CGM system 104 includes light emitting diodes (LEDs) which are implemented to transmit light directed at skin 206 disposed around the sensor's 202 insertion site. The light transmitted by the LEDs reflects from the skin 206 disposed around the sensor's 202 insertion site, and this reflected light is received by the photodiode sensor. The CGM system 104 generates the light data as describing light reflected from the skin 206 disposed around the sensor's 202 insertion site.

The adaptive system 314 (and/or the computing device 108) processes the light data to identify the anomaly. To do so, the computing device 108 compares reflected light patterns described by the light data with characteristic light patterns indicative of an anomaly of the sensor's 202 insertion site. For example, the anomaly of the insertion site is a tattoo, a scar tissue, a skin irritation, etc. The computing device 108 identifies the anomaly as corresponding to most similar characteristic light pattern to a light pattern described by the light data. Once identified, the computing device 108 determines amounts by which the glucose measurements 118 should be increased or decreased based on the anomaly of the insertion site. The adaptive system 314 generates the modified data 316 as describing the glucose measurements 118 that are increased or decreased by the determined amounts.

Consider an example in which the adaptive system 314 (and/or the computing device 108) is implemented to generate the modified data 316 based on heart rate data. In this example, the heart rate data describes the person's 102 heart rate, heart rate variability, oxygen saturation, etc. As used herein, the term “heart rate variability” refers to variations in time intervals between heartbeats and these variations can indicate corresponding variations in the person's 102 blood glucose levels. For example, the non-glucose data 310 includes the heart rate data and the adaptive system 314 (and/or the computing device 108) processes the glucose data 308, the non-glucose data 310, and/or the other data 312 to generate the modified data 316. In one example, the adaptive system 314 (and/or the computing device 108) uses the heart rate data to determine a modification amount by which a particular user glucose value described by the glucose data 308 should be increased or decreased to improve an accuracy of the particular user glucose value.

Consider an example in which the adaptive system 314 (and/or the computing device 108) leverages historic heart rate data and historic glucose data to form at least one model to improve the accuracy of the particular user glucose value. Since the user glucose values are representative of localized blood glucose concentrations in the person 102 and because the person's 102 heart circulates the person's 102 blood as it beats over time, the user glucose values are at least partially dependent on the person's 102 heartbeats. For example, a glucose measurement 118 from the person's 102 interstitial fluid at a particular time may correspond to a localized glucose concentration in the person's 102 blood about 10 minutes before the particular time.

### Probabilistic Models

In one example, the adaptive system 314 (and/or the computing device 108) forms a probabilistic model using the historic heart rate data and the historic glucose data such that for any particular observed heart rate value at a first time, the probabilistic model outputs a probability of observing a particular user glucose value at a second time based on the historic data. The first time is before the second time. In an example, the historic heart rate data is historic heart rate data of the person 102 and the historic glucose data is historic glucose data of the person 102. In another example, the historic heart rate data is historic heart rate data of the user population 110 and the historic glucose data is historic glucose data of the user population 110. In some examples, the adaptive system 314 (and/or the computing device 108) forms the probabilistic model such that the model outputs a probability (e.g., and a confidence level) of observing a particular user glucose value at the second time based on an observation of multiple heart rate values at the first time.

In a first example, the adaptive system 314 receives the non-glucose data 310 which includes the heart rate data describing the person's 102 heart rate and the adaptive system 314 extracts a user heart rate value (e.g., 70 beats per minute) at a first time from the heart rate data. In this example, the adaptive system 314 (and/or the computing device 108) uses the user heart rate value as an input to the probabilistic model which outputs a mostly likely user glucose value (e.g., 125 mg/dL) to be observed at a second time. For example, the first time is 9:00 AM and the second time is 9:15 AM. In some examples, the probabilistic model also outputs a confidence level such as a 95% confidence of an observed user glucose value equal to 125 mg/dL at the second time based on the historic heart rate data and the historic glucose data.

Continuing the first example, the adaptive system 314 receives the glucose data 308 describing user glucose values measured by the CGM system 104. The adaptive system 314 (and/or the computing device 108) identifies a particular user glucose value described by the glucose data 308 having a timestamp corresponding to 9:15 AM. For example, the particular user glucose value is 166 mg/dL which is significantly different from the predicted user glucose value of 125 mg/dL. In an example, the adaptive system 314 (and/or the computing device 108) leverages the probabilistic model using the user heart rate value and the particular user glucose value as inputs to determine a probability of observing the particular user glucose value at the second time based on the historic heart rate data and the historic glucose data. In this example, the probabilistic model outputs a probability of less than one percent of observing 166 mg/dL at the second time with a 95% confidence level.

In the first example, the adaptive system 314 determines a modification amount equal to 41 mg/dL which corresponds to an amount by which the particular user glucose value should be reduced based on the historic heart rate data and the historic glucose data. The adaptive system 314 modifies the particular user glucose value by the modification amount and generates the modified data 316 as describing a modified particular user glucose value. In one example, the adaptive system 314 generates the indication 318 to indicate the modified particular user glucose value. In another example, the adaptive system 314 generates the indication 318 to indicate how the glucose data 308 was modified to generate the modified data 316.

In a second example, the adaptive system 314 forms the probabilistic model based on multiple measured values described by the historic heart rate data. For example, based on the historic heart rate data and the historic glucose data, the adaptive system 314 (and/or the computing device 108) forms the probabilistic model such that for inputs of a heart rate value and a heart rate variability value at a first time, the model outputs a probability of observing a user glucose value at a second time. In this second example, forming the probabilistic model based on the multiple measured values described by the historic heart rate data increases accuracy of the model.

In a third example, the adaptive system 314 (and/or the computing device 108) forms the probabilistic model based on values described by the historic heart rate data and values described by the historic glucose data. In this example, the adaptive system 314 (and/or the computing device 108) forms the probabilistic model such that for inputs of a heart rate value and a user glucose value at a first time, the probabilistic model outputs a probability of observing a user glucose value at a second time. Similar to the second example, forming the probabilistic model based on the values described by the historic heart rate data and the values described by the historic glucose data also increases accuracy of the model.

Consider an example in which, in addition to including the heart rate data, the non-glucose data 310 also includes perspiration data describing increases or decreases in amounts of the person's 102 perspiration over time. In an example, the adaptive system 314 (and/or the computing device 108) leverages the perspiration data in a manner that is independent of the heart rate data. For example, the perspiration data describes measured sweat glucose values of the person 102 over time and the adaptive system 314 converts the sweat glucose values into equivalent blood glucose values. In this example, the adaptive system 314 compares an equivalent blood glucose value corresponding to a particular time to a user glucose value corresponding to the particular time.

If a difference between the equivalent blood glucose value and the user glucose value is smaller than a threshold difference, then the adaptive system 314 converts a next measured sweat glucose value described by the perspiration data into an additional equivalent blood glucose value which the adaptive system 314 compares to a next user glucose value. In a first example in which the difference between the equivalent blood glucose value and the user glucose value is greater than the difference threshold, the adaptive system 314 generates the indication 318 to indicate that the equivalent blood glucose value and the user glucose value are significantly different. In a second example in which the difference between the equivalent blood glucose value and the user glucose value is greater than the difference threshold, the adaptive system 314 (and/or the computing device 108) leverages the probabilistic model and the heart rate data to determine a probability (e.g., and a confidence level) of observing the user glucose value at the particular time.

If the probability of observing the blood glucose value at the particular time is low and a corresponding confidence level in the probability is high, then the adaptive system 314 (and/or the computing device 108) leverages the probabilistic model to determine a particular user glucose value which is most likely to be observed at the particular time based on the historic heart rate data and the historic glucose data. For example, the adaptive system 314 determines a difference between the particular user glucose value and the user glucose value and compares this determined difference to a second threshold. If the determined difference is less than the second threshold, then the adaptive system generates the indication 318 to indicate that the equivalent blood glucose value and the user glucose value are significantly different. If the determined difference is greater than the second threshold, then the adaptive system 314 (and/or the computing device 108) implements the probabilistic model to determine a probability (e.g., and a confidence level) of observing the particular user glucose value at the particular time.

If the probability of observing the particular user glucose value at the particular time is relatively low and associated with a relatively high confidence level, then the adaptive system 314 generates the indication 318 to indicate that the equivalent blood glucose value and the user glucose value are significantly different. If the probability of observing the particular user glucose value at the particular time is relatively high and associated with a relatively high confidence level, then the adaptive system 314 (and/or the computing device 108) determines a modification amount by which to modify the user glucose value based on the historic heart rate data and the historic glucose data. The adaptive system 314 modifies the user glucose value by the determined modification amount and generates the modified data 316 as describing the modified user glucose value. For example, the adaptive system 314 generates the indication 318 to communicate how the glucose data 308 is modified to generate the modified data 316.

In some examples, rather than describing measured sweat glucose values of the person 102 over time, the perspiration data describes increases and decreases in amounts of the person's 102 perspiration over time. In these examples, the adaptive system 314 (and/or the computing device 108) leverages the perspiration data as a screening tool to determine whether or not to implement the probabilistic model. For example, the probabilistic model is computationally expensive in some implementations and the adaptive system 314 (and/or the computing device 108) uses trends described by the perspiration data to screen the glucose data 308 for potential inaccuracies which is computationally inexpensive relative to an implementation of the probabilistic model.

In one example, the adaptive system 314 (and/or the computing device 108) processes the perspiration data and identifies a temporal window in which perspiration values corresponding to amounts of the person's 102 perspiration are increasing. In general, the increasing perspiration values can correspond to increasing user glucose values described by the glucose data 308. The adaptive system 314 determines a modified temporal window for screening the glucose data 308 based on the temporal window. For example, there may be temporal delay between the increasing perspiration values of the person 102 and the corresponding increasing user glucose values described by the glucose data 308, and the adaptive system 314 (and/or the computing device 108) determines the modified temporal window based on the temporal delay.

The adaptive system 314 (and/or the computing device 108) determines a subset of the user glucose values described by the glucose data 308 using the modified temporal window and then determines whether user glucose values included in the subset are generally increasing. If the adaptive system 314 (and/or the computing device 108) determines that the user glucose values included in the subset are generally increasing, then the adaptive system 314 concludes that the user glucose values included in the subset are likely accurate and processes the perspiration data to identify an additional temporal window in which the perspiration values corresponding to amounts of the person's 102 perspiration are increasing. If the adaptive system 314 (and/or the computing device 108) determines that the user glucose values included in the subset are not generally increasing (e.g., the user glucose values included in the subset are decreasing), then the adaptive system 314 determines that the user glucose values included in the subset are likely not accurate. Based on determining that the user glucose values included in the subset are likely not accurate, the adaptive system 314 (and/or the computing device 108) implements the probabilistic model to determine probabilities of observing the user glucose values included in the subset based on the historic heart rate data and the historic glucose data as previously described.

Consider an additional example in which the adaptive system 314 leverages the non-glucose data 310 as part of a tool for screening the glucose data 308 and/or as a basis for forming the probabilistic model. In this additional example, the non-glucose data 310 describes the person's 102 physical activities. For example, the person 102 interacts with a user interface of the computing device 108 to specify specific activities completed by the person 102 in the past and/or planned for completion in the future by the person 102. The computing device 108 generates activity data describing the specific activities completed and/or planned for completion which is included in the CGM device data 214 and/or included in the non-glucose data 310.

In another example, the CGM system 104 generates the activity data, for example, using an accelerometer included in the additional sensors 220. In this other example, the accelerometer measures forces, e.g., due to movements of the person 102. The sensor module 204 receives communications describing the measured forces from the accelerometer, and the sensor module 204 generates the activity data as describing steps taken by the person 102 over time.

Consider an example in which the computing device 108 includes an accelerometer that measures forces caused by movements of the person 102. For example, an activity module of the computing device 108 receives communications from the accelerometer describing the measured forces, and the activity module processes the communications to generate the activity data describing steps taken by the person 102 over time. In this example, the activity data is included in the CGM device data 214 and/or included in the non-glucose data 310.

In one example, the adaptive system 314 (and/or the computing device 108) processes the activity data describing the steps taken by the person 102 over time to identify a temporal window within which the steps taken by the person 102 (or an absence of steps taken by the person 102) corresponds to a scenario that is likely to affect the person's 102 blood glucose levels. For example, many steps taken within a short period of time is likely indicative of an exercise activity. Exercise generally lowers the person's 102 blood glucose levels; however, very intense physical activity over a relatively short period of time can cause the person's 102 blood glucose levels to spike and then decrease which may continue for several hours after the person 102 completes the exercise activity.

An absence of steps taken by the person 102 within a relatively long period of time is likely indicative of a sleep cycle. Sleeping generally lowers the person's 102 blood glucose levels or results in stable glucose levels; however, the person's 102 blood glucose levels generally increase near an end of the sleep cycle. In some examples, the adaptive system 314 leverages timestamps included in the activity data to determine whether the person 102 is likely sleeping and/or when an increase in the person's 102 blood glucose levels near the end of a sleep cycle is likely to occur.

After the adaptive system 314 (and/or the computing device 108) identifies a temporal window within which the activity data is indicative of a scenario that is likely to affect the person's 102 blood glucose levels, the adaptive system 314 (and/or the computing device 108) approximates a temporal delay between a time corresponding to an end of the temporal window and a time at which the glucose measurements 118 begin to reflect the person's 102 activity within the temporal window. The adaptive system 314 (and/or the computing device 108) determines a modified temporal window for screening the glucose data 308 based on the temporal delay. For example, the adaptive system 314 (and/or the computing device 108) determines a subset of the user glucose values described by the glucose data 308 using the modified temporal window and then determines whether user glucose values included in the subset correspond to the person's 102 steps or lack of steps included in the temporal window.

If the adaptive system 314 (and/or the computing device 108) determines that the user glucose values included in the subset correspond to the person's 102 steps or lack of steps included in the temporal window, then the adaptive system 314 determines that the user glucose values included in the subset are likely accurate. Upon concluding that the user glucose values included in the subset are likely accurate, the adaptive system 314 continues to process the activity data to identify an additional temporal window within which the steps taken by the person 102 (or lack of steps taken by the person 102) correspond to a scenario that is likely to affect the person's 102 blood glucose levels. If the adaptive system 314 (and/or the computing device 108) determines that the user glucose values included in the subset do not correspond to the person's 102 steps or lack of steps included in the temporal window, then the adaptive system 314 determines that the user glucose values included in the subset are likely not accurate. In response to determining that the user glucose values included in the subset are likely not accurate, the adaptive system 314 (and/or the computing device 108) implements the probabilistic model to determine probabilities of observing the user glucose values included in the subset based on the historic heart rate data and the historic glucose data as described previously.

In one example, the adaptive system 314 (and/or the computing device 108) forms the probabilistic model based on historic activity data, the historic heart rate data, and the historic glucose data. In this example, an observed heart rate value at a first time and an observed temporal window including steps taken by the person 102 at the first time are combined as inputs to the probabilistic model which outputs a probability of observing a particular user glucose value at a second time based on the historic data. By forming the probabilistic model from the historic activity data in addition to the historic heart rate data, the adaptive system 314 increases an accuracy of the probabilistic model.

### Machine Learning Models

In some examples, the adaptive system 314 (and/or the computing device 108) leverages stress data describing levels of stress experienced by the person 102 over time and/or mood data describing the person's 102 mood over time as part of a screening tool to screen the glucose data 308. For example, the computing device 108 includes an image capture device which captures digital images of the person 102 (e.g., depicting the person's 102 face). The computing device 108 implements a machine learning model trained using training data to classify a mood of the person 102 from an input digital image depicting the person's 102 face. In this example, the machine learning model is also trained using training data to quantify a level of stress experienced by the person 102 from the input digital image depicting the person's 102 face and the computing device 108 implements the machine learning model to quantify the level of stress experienced by the person 102. For example, the training data includes digital images of faces and the machine learning model learns to classify moods and quantify levels of stress based on features depicted in the digital images of faces.

The computing device 108 generates the stress data and/or the mood data based on outputs from the machine learning model and the computing device 108 includes the stress data and/or the mood data in the CGM package 302 and/or the non-glucose data 310. The adaptive system 314 receives the non-glucose data 310 which includes the stress data and/or the mood data, and the adaptive system 314 processes the stress data and/or the mood data to screen the glucose data 308 for accuracy as previously described. For example, the adaptive system 314 (and/or the computing device 108) identifies a subset of the stress data and/or the mood data which corresponds to a scenario likely to affect the person's 102 blood glucose levels. The adaptive system 314 (and/or the computing device 108) uses the subset of the stress data and/or the mood data along with corresponding temporal delays to screen the glucose data 308. Based on this screening, the adaptive system 314 determines whether or not to implement the probabilistic model.

Consider an example in which the computing device 108 implements a machine learning model to identify the location 124 of the sensor's 202 insertion site. In this example, the machine learning model is trained on training data describing characteristic force patterns that are each associated with a possible location of the sensor's 202 insertion site. Thus, the machine learning model learns to classify insertion site locations based on the training data and the training. For example, the computing device 108 formats the orientation data 304 in a format configured for processing by the machine learning model. The machine learning model receives the orientation data 304 in the format and processes the formatted orientation data 304 to generate an indication of the location 124.

In one example, the adaptive system 314 leverages acquisition data describing food acquired by the person 102 over time and/or consumption data describing food consumed by the person 102 over time as a screening tool for the glucose data 308. For example, the consumption data includes event data describing carbohydrates consumed by a user of the CGM system 104. In this example, the computing device 108 generates the acquisition data and/or the consumption data.

For example, the computing device 108 receives inputs from the person 102 describing food acquired and food consumed and the computing device 108 generates the acquisition data and/or the consumption data based on these inputs. In an example, the computing device 108 includes the acquisition data and/or the consumption data in the CGM package 302 and/or the non-glucose data 310. The adaptive system 314 receives the non-glucose data 310 and processes the acquisition data and/or the consumption data to screen the glucose data 308 as described above.

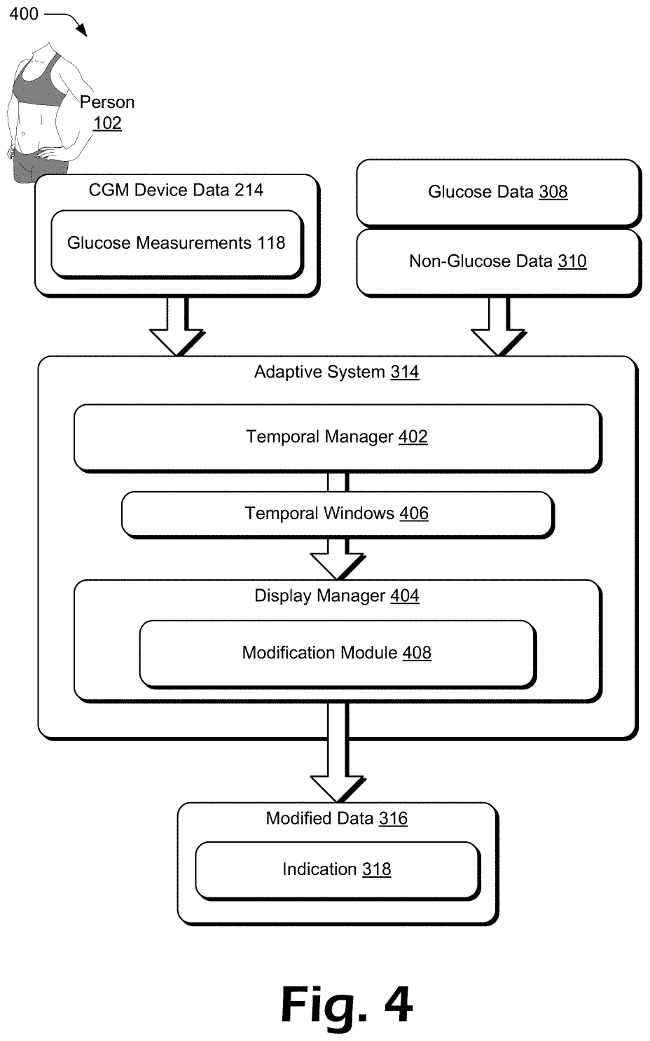

FIG. 4 depicts an example 400 implementation of the adaptive system 314 of FIG. 3 in greater detail. The adaptive system 314 is illustrated to include a temporal manager 402 and a display manager 404. As shown, the adaptive system 314 receives the glucose data 308 and the non-glucose data 310 as inputs. The adaptive system 314 is also illustrated as receiving the CGM device data 214 which includes the glucose measurements 118. In some examples, the adaptive system 314 generates the glucose data 308 and the non-glucose data 310 based on the CGM device data 214.

### Temporal Windows

The temporal manager 402 receives the glucose data 308 and the non-glucose data 310 and processes the glucose data 308 and/or the non-glucose data 310 to generate temporal windows 406. The temporal windows 406 each define a beginning and an end of a timeseries of values described by the glucose data 308 and/or the non-glucose data 310. The adaptive system 314 implements the temporal manager 402 to generate the temporal windows 406 as part of exposing a variety of functionalities.

Consider an example in which the adaptive system 314 implements the temporal manager 402 to generate the temporal windows 406 as part of preparing a glucose value report. In this example, the glucose value report includes a summary of the person's 102 glucose measurements 118 over a time period beginning when the person 102 installs a single-use glucose sensor in the CGM system 104 and ending when the person 102 uninstalls the single-use glucose sensor from the CGM system 104 in order to install a new single-use glucose sensor in the CGM system 104. For example, the adaptive system 314 implements the temporal manager 402 to generate a first temporal window which begins when the single-use glucose sensor is installed in the CGM system 104 and ends at a time corresponding to a timestamp of a most recent user glucose value described by the glucose data 308. The first temporal window defines a session and the temporal manager 402 generates a second temporal window that begins when the single-use glucose sensor is installed in the CGM system 104 and ends one day (e.g., 24 hours) after the single-use glucose sensor is installed in the CGM system 104.

The second temporal window defines an undesirable period during the session in which inaccuracies in the glucose data 308 such as compression artifacts are more likely to occur than during a remaining portion of the session. These inaccuracies in the glucose data 308 are due in part to the “cold start” nature of the undesirable period. For example, including the undesirable period in the glucose value report causes the summary of the person's 102 glucose measurements 118 during the session to be inaccurate due to the inaccuracies of the undesirable period.

In one example, the adaptive system 314 leverages the second temporal window to remove the undesirable period from the session. In this example, the adaptive system 314 then implements the temporal manager 402 to generate a third temporal window that begins when the second temporal window ends. This third temporal window ends at the end of the first temporal window or the time corresponding to the timestamp of the most recent user glucose value described by the glucose data 308. For example, the adaptive system 314 uses glucose measurements included in the third temporal window to prepare the glucose value report which has improved accuracy due to the omission of the undesirable period.

Consider an example in which the adaptive system 314 implements the temporal manager 402 to generate the temporal windows 406 as part of screening the glucose data 308 for inaccuracies. In this example, the temporal manager 402 generates the temporal windows 406 to correlate timeseries data included in the non-glucose data 310 with timeseries data included in the glucose data 308. For example, the non-glucose data 310 includes activity data describing steps taken by the person 102 over time. The adaptive system 314 processes the activity data to identify a scenario which is likely to affect the person's 102 blood glucose levels.

In one example, the adaptive system 314 (and/or the computing device 108) identifies a period of time described by the activity data having a beginning and an end. The activity data describes many steps taken by the person 102 during the period of time and the adaptive system 314 determines that a number of steps taken by the person 102 during the period of time corresponds to an exercise activity. The adaptive system 314 implements the temporal manager 402 to generate a temporal window that begins at the beginning of the period of time and ends at the end of the period of time.

For example, the adaptive system 314 (and/or the computing device 108) determines a temporal delay which corresponds to a period of time between an occurrence of the exercise activity and a time when the glucose measurements 118 reflect changes in the person's 102 blood glucose levels that are a result of the exercise activity. The temporal delay can include multiple components in some examples. In this example, the person's 102 blood glucose levels may decrease because of the exercise activity for hours after the person 102 completes the exercise activity which is a first component of the temporal delay. In an example in which the sensor 202 takes the glucose measurements 118 from interstitial fluid of the person 102, there can be a delay of about 10 minutes after a change in the person's 102 blood glucose concentrations before a corresponding change in the person's 102 interstitial fluid glucose concentrations which is a second component of the temporal delay.

After determining the temporal delay, the adaptive system 314 implements the temporal manager 402 to generate a modified temporal window based on the temporal delay. For example, the temporal manager 402 generates the modified temporal window by shifting the temporal window in time by the temporal delay. The adaptive system 314 applies the modified temporal window to the glucose data 308 and determines a subset of the user glucose values described by the glucose data 308. For example, user glucose values included in the subset are included within the modified temporal window.

As illustrated in FIG. 4, the display manager 404 receives the temporal windows 406 which include the modified temporal window defining the subset of the user glucose values described by the glucose data 308. The display manager 404 processes the user glucose values included in the subset to determine whether these values reflect the exercise activity. If the display manager 404 determines that the user glucose values included in the subset do reflect the exercise activity, then the display manager 404 processes data defined by another temporal window included in the temporal windows 406.

If the display manager 404 determines that the user glucose values included in the subset do not reflect the exercise activity, then the display manager 404 may perform a variety of different procedures to evaluate an accuracy of the user glucose values included in the subset. For example, the display manager 404 (and/or the computing device 108) implements the probabilistic model to determine a probably of observing the user glucose values included in the subset based on the non-glucose data 310 and historic glucose data, most likely user glucose values to observe based on the non-glucose data 310 and historic glucose data, and so forth. In an example in which the display manager 404 determines that a user glucose value included in the subset is not accurate and should be modified, the display manager 404 implements a modification module 408 to generate the modified data 316 and/or the indication 318.

For example, the modification module 408 modifies the user glucose value included in the subset that is not accurate by generating a modified user glucose value having improved accuracy relative to the user glucose value. The modification module 408 generates the modified data 316 as describing the modified user glucose value. In one example, the modification module 408 generates the indication 318 as describing how the glucose data 308 was modified to generate the modified data 316. The computing device 108 receives the modified data 316 and the indication 318, and the computing device 108 processes the modified data 316 and/or the indication 318 to display the indication 318 in a user interface of the computing device 108.

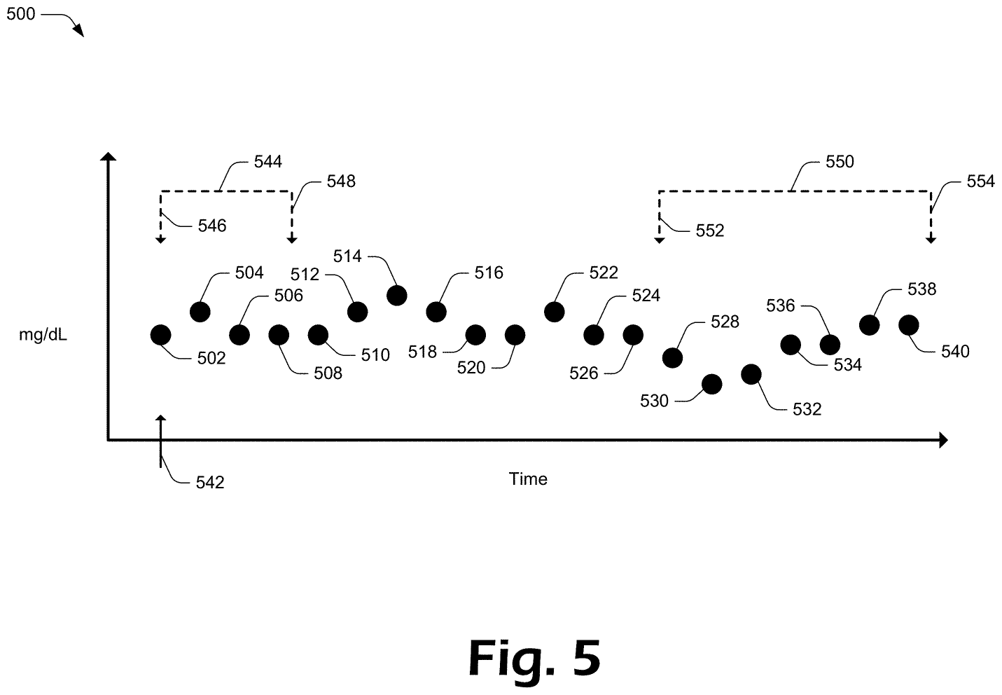

FIG. 5 illustrates a representation 500 of session data describing historic user glucose values measured by a single-use glucose sensor since the single-use glucose sensor was installed in a continuous glucose monitoring (CGM) system. As shown, the representation 500 includes user glucose values 502-540 which are measured by a single-use glucose sensor of the CGM system 104 that is worn by the person 102. For example, the user glucose values 502-540 vary over time as the person's 102 blood glucose level varies over time. The representation 500 also includes an indication 542 which corresponds to an installation of the single-use glucose sensor in the CGM system 104.

As illustrated, glucose value 502 corresponds to a first glucose measurement 118 after the installation of the single-use glucose sensor in the CGM system 104. As described previously, the glucose value 502 has a higher probability of inaccuracy than, for example, glucose value 522 because of the “cold start” scenario when the single-use glucose sensor is installed. Further, the “cold start” creates an undesirable period lasting about one day after the indication 542. During this undesirable period, the glucose measurements 118 have a higher probability of corresponding to an inaccurate one of the glucose values 502-508.

A first temporal window 544 defines the undesirable period. As shown, the first temporal window 544 has a beginning 546 and an end 548. The beginning 546 corresponds to the indication 542 and the end 548 corresponds to a time approximately 24 hours from the beginning 546. It is to be appreciated that the undesirable period may be less than a 24 hour time period. In some examples, the undesirable period is 3 hours, 6 hours, 9 hours, 12 hours, 15 hours, 18 hours, and so forth. It is also to be appreciated that the undesirable period may be greater than 24 hours as well such as 30 hours, 36 hours, 42 hours, 48 hours, etc. In an example, the undesirable period is expressed as a percentage of a session such as a first 10 percent of the session.

The representation 500 also includes a second temporal window 550 having a beginning 552 and an end 554. The second temporal window 550 includes user glucose value 540 which is a most recent user glucose value described by the glucose data 308. For example, the second temporal window 550 includes user glucose values 528-540 and no portion of the second temporal window 550 overlaps a portion of the first temporal window 544. Accordingly, the user glucose values 528-540 do not suffer from the increased probability of inaccuracy associated with the user glucose values 502-508 which are included in the first temporal window 544.

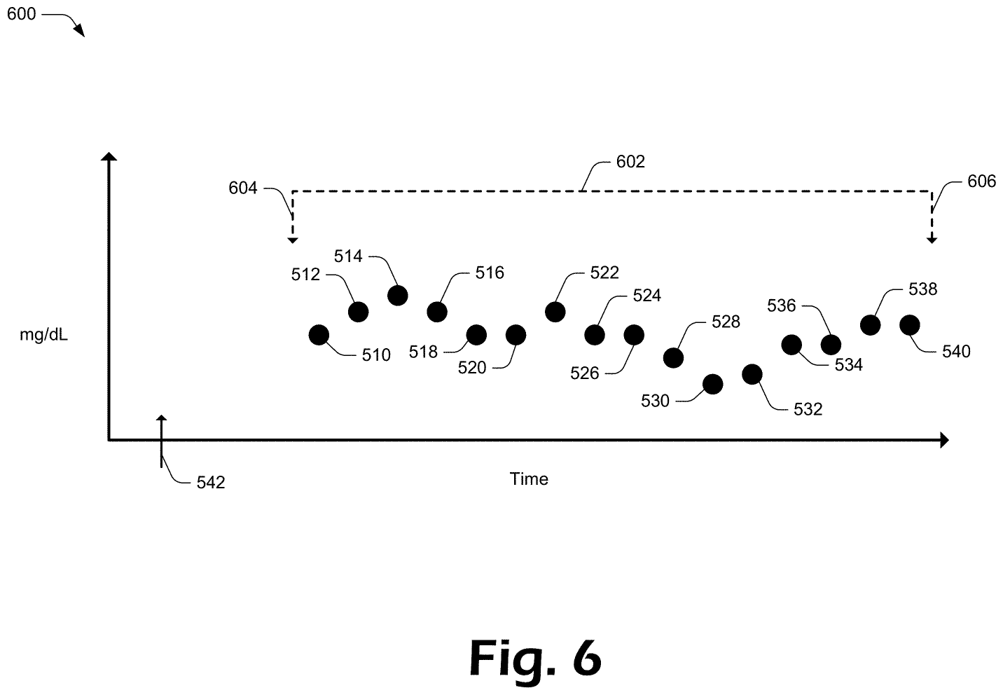

FIG. 6 illustrates a representation 600 of modified session data usable to generate a glucose value report. As illustrated, the representation 600 includes user glucose values 510-540 and the representation 600 does not include user glucose values 502-508. For example, a healthcare provider for the person 102 receives the glucose value report and uses information included in the glucose value report as a decision-making guide for managing the person's 102 blood glucose levels.

Due to the clinical significance of the glucose value report, the adaptive system 314 (and/or the computing device 108) excludes the user glucose values 502-508 from a session window 602. The session window 602 has a beginning 604 and an end 606. In the illustrated example, the beginning 604 corresponds to the end 548 of the first temporal window 544 that defines the undesirable period. The end 606 corresponds to the end 554 of the second temporal window 550. For example, the adaptive system 314 uses the user glucose values 510-540 included in the session window 602 to generate the glucose value report.

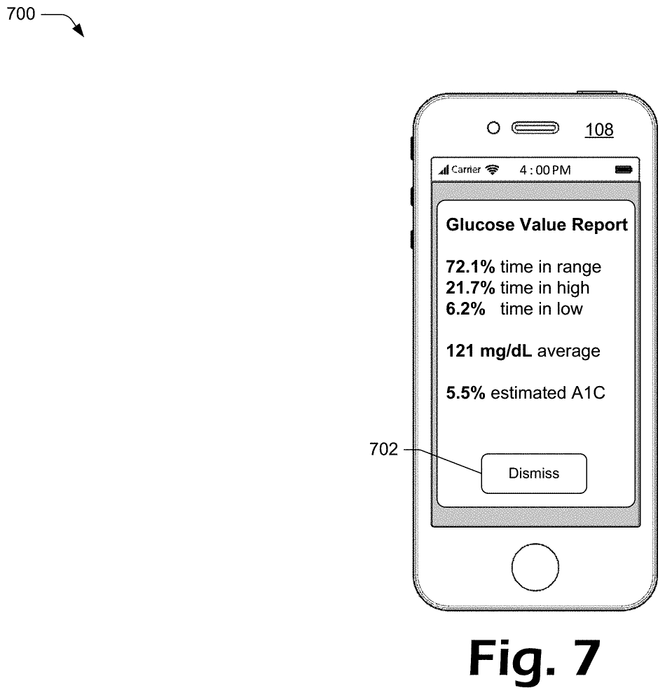

FIG. 7 illustrates a representation 700 of a glucose value report displayed in a user interface of a computing device. As shown, the representation 700 includes the computing device 108 which is illustrated as a smartphone that the person 102 uses to display the glucose value report for a healthcare provider. In some examples, the computing device 108 is the healthcare provider's computing device 108 and the healthcare provider receives the glucose value report via the network 116. In the illustrated example, the adaptive system 314 generates the glucose value report from the user glucose values 510-540 included in the session window 602.

The glucose value report indicates that the person's 102 blood glucose levels were in range 72.1 percent of the time between the beginning 604 and the end 606 of the session window 602. The glucose value report also indicates that the person's 102 blood glucose levels were high 21.7 percent of the time and low 6.2 percent of the time during the session. An average value of the user glucose values 510-540 is 121 mg/dL and the person's 102 estimated A1C is 5.5 percent based on data included in the session window 602.

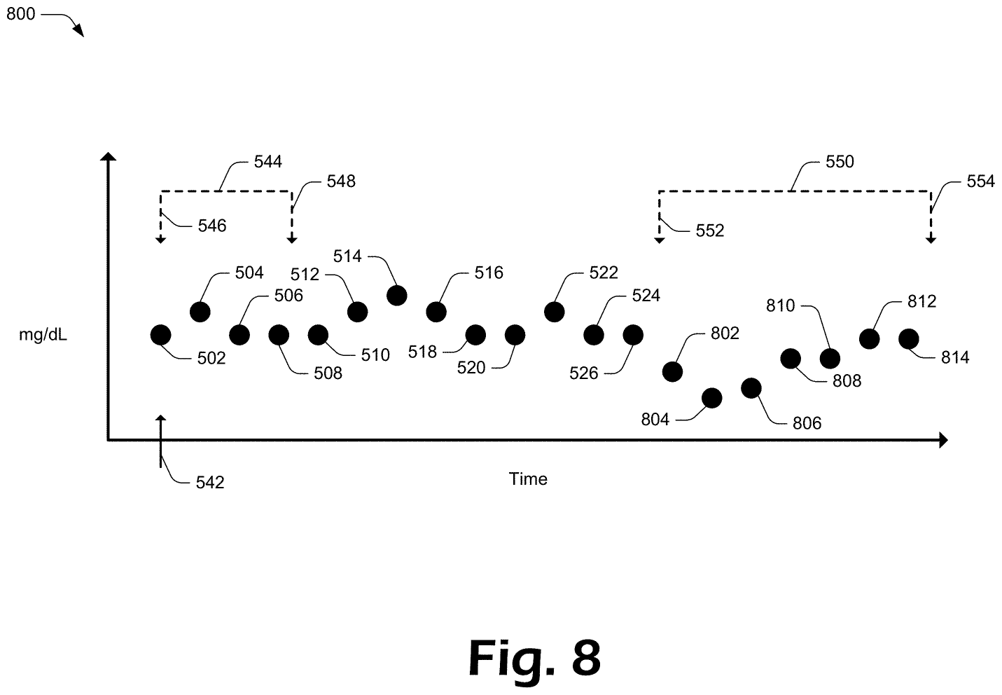

FIG. 8 illustrates a representation 800 of glucose data and modified glucose data. For example, the glucose data 308 includes the glucose values 502-540 depicted in the representation 500. As shown, the representation 800 includes the glucose values 502-526 which is a subset of the glucose values 502-540 described by the glucose data 308. The representation 800 also includes glucose values 802-814 described by the modified glucose data. For example, the glucose values 528-540 included in the representation 500 are replaced by glucose the values 802-814, respectively, in the representation 800. In the illustrated example, the adaptive system 314 (and/or the computing device 108) generates the modified glucose data by replacing the glucose values 528-540 with the glucose values 802-814.

Consider a first example in which the person 102 installs the CGM system 104 at a time indicated by the indication 542. In this example, the person 102 attaches the CGM system 104 to the person's 102 thigh which is not an indicated location for wearing the CGM system 104. Accordingly, the location of the sensor's 202 insertion site is the person's 102 thigh. Thus, in this example, the person 102 is using the CGM system 104 in a manner which conflicts with instructions for using the CGM system 104. As a result of this, the sensor 202 is disposed below the skin 206 of the person 102 in an anatomical location that is different from anatomical locations corresponding to an intended use of the CGM system 104. These differences can adversely affect accuracy of the glucose measurements 118 in some examples.

The sensor 202 takes glucose measurements 118 in the worn location of the CGM system 104 while the sensor 202 is inserted on the person's 102 thigh. The glucose data 308 describes the glucose values 502-526 which correspond to the glucose measurements 118 taken by the sensor 202. For example, an accelerometer of the additional sensors 220 measures forces while the person 102 wears the CGM system 104. These forces are caused by movements of the person 102, and the CGM system 104 generates orientation data 304 as describing the forces measured by the accelerometer.

The adaptive system 314 (and/or the computing device 108) receives the glucose data 308 and also the non-glucose data 310 which includes the orientation data 304 in this example. In one example, the adaptive system 314 processes the orientation data 304 to identify the location of the sensor's 202 insertion site. To do so, the adaptive system 314 compares the forces described by the orientation data 304 with characteristic force patterns that each correspond to a location on the person 102 in which it is possible to insert the sensor 202.

For example, the accelerometer of the CGM system 104 experiences different forces when the sensor 202 is inserted at different locations on the person 102. Because of these differences, each location in which it is possible to insert the sensor 202 on the person 102 can be uniquely identified based on its corresponding characteristic force pattern. By leveraging the characteristic force patterns in this way, the adaptive system 314 (and/or the computing device 108) identifies the location of the sensor's 202 insertion site based on similarities between the forces described by the orientation data 304 and a characteristic force pattern that corresponds to the location of the sensor's 202 insertion site on the person's 102 thigh.

For example, the adaptive system 314 initially compares the forces described by the orientation data 304 with characteristic force patterns that correspond to intended locations for inserting the sensor 202 such as the person's 102 buttock or abdomen. Based on this initial comparison, the adaptive system 314 (and/or the computing device 108) determines that the location of the sensor's 202 insertion site is not on the person's 102 arm or the person's 102 abdomen. In one example, the adaptive system 314 generates an alert for display in a user interface of the computing device 108 that indicates to the person 102 that sensor's 202 insertion site location is not an intended location for inserting the sensor 202. For example, this alert also indicates that the person's 102 misuse may affect an accuracy of glucose measurements 118 taken by the CGM system 104.

In another example, the adaptive system 314 (and/or the computing device 108) compares the forces described by the orientation data 304 with characteristic force patterns of possible insertion site locations for the sensor 202 (other than the person's 102 abdomen or arm). The adaptive system 314 identifies the characteristic force pattern that corresponds to the sensor's 202 insertion site location on the person's 102 thigh as being a most similar one of the characteristic force patterns to the forces described by the orientation data 304. Accordingly, the adaptive system 314 identifies the sensor's 202 insertion site location as being the thigh of the person 102. In some examples, the adaptive system 314 generates a confirmation request for the person 102 to confirm whether or not the CGM system 104 is worn on the person's 102 thigh. An example of this is described in greater detail with respect to FIG. 9.

In other examples, the adaptive system 314 (and/or the computing device 108) determines a risk of the person 102 wearing the CGM system 104 while the sensor's 202 insertion site location is on the person's 102 thigh. In some examples, each of the locations on the person 102 in which it is possible to insert the sensor 202 (e.g., other than the intended locations) is classified based on risk. For example, these classifications include low risk, moderate risk, and high risk. In general, a risk of injury to the person 102 is greater if the person 102 is wearing the CGM system 104 in a manner in which the sensor's 202 insertion site location is a high risk location than if the person 102 is wearing the CGM system 104 such that the sensor's 202 insertion site location is in a moderate risk location. Similarly, the risk of injury to the person 102 is greater if the person 102 is wearing the CGM system 104 with the location of the sensor's 202 insertion site in a moderate risk location than if the person 102 is wearing the CGM system 104 with the location of the sensor's 202 insertion site in a low risk location.

For sensor 202 insertion site locations classified as low risk, the adaptive system 314 performs minimal intervention. For example, the adaptive system 314 generates the confirmation request for a low risk sensor 202 insertion site location. For sensor 202 insertion site locations classified as moderate risk, the adaptive system 314 performs moderate intervention such as generating an alarm for the person 102 to communicate the risk.

In some examples, the adaptive system 314 (and/or the computing device 108) generates an alert for the person's 102 healthcare provider for moderate risk sensor 202 insertion site locations. In an example, the adaptive system 314 performs substantial intervention for sensor 202 insertion site locations classified as high risk such as generating multiple alarms for the person 102 and/or generating a confirmation request for the person 102 to confirm that the sensor's 202 insertion site is no longer in the high risk location. This substantial intervention can include generating an alarm for the person's 102 healthcare provider indicating a high risk to the person 102 based on the sensor's 202 insertion site location.

In the illustrated example, the adaptive system 314 (and/or the computing device 108) determines that the sensor's 202 insertion site location on the person's 102 thigh corresponds to a low risk of injury to the person 102. Accordingly, the adaptive system generates the confirmation request for the person 102 to confirm whether or not the sensor's 202 insertion site location is on the person's 102 thigh. In an example in which the adaptive system 314 receives data describing an interaction by the person 102 with a user interface of the computing device 108 in which the person 102 indicates that the location of the sensor's 202 insertion site is not on the person's 102 thigh, the adaptive system 314 may not generate the modified glucose data.

However, in an example in which the adaptive system 314 receives data describing an interaction by the person 102 with the user interface of the computing device 108 in which the person 102 confirms that the location of the sensor's 202 insertion site is on the person's 102 thigh, then the adaptive system 314 (and/or the computing device 108) may generate the modified glucose data. For example, the adaptive system 314 determines whether or not to generate the modified glucose data by estimating an effect of the location of the sensor's 202 insertion site on the glucose values 502-540. In one example, the adaptive system 314 determines differences between the glucose values 502-540 and ideal glucose values to estimate the effect of the location of the sensor's 202 insertion site.

Consider an example in which the adaptive system 314 determines a difference between the glucose values 502-540 and ideal glucose values which would be measured by the CGM system 104 if the location of the sensor's 202 insertion site was on the person's 102 abdomen or buttock. For example, the adaptive system 314 accesses sensor 202 insertion site location conversion data that describes modification values usable to convert glucose values of glucose measurements 118 taken from a first location of the sensor's 202 insertion site to glucose values of glucose measurements 118 taken from a second location of the sensor's 202 insertion site. In some examples, the modification values are determined theoretically, for example, the modification values are calculated based on differences between each of the possible insertion site locations for the sensor 202 on the person 102. The differences can include bioelectrical differences, dimensional differences, fluidic differences, and so forth.

In other examples, the modification values are determined analytically such as by the person 102 or a similar person simultaneously wearing multiple CGM systems 104 with sensors 202 inserted at different insertion site locations. Glucose measurements 118 taken at the same time but with sensors 202 in different insertion site locations of the person 102 are then compared to determine the modification values. For example, the differences between glucose measurements 118 from the sensors 202 in the different insertion site locations on the person 102 are used as training data for a machine learning model.

In this example, the machine learning model is trained to generate ideal glucose measurements based on the training data. In one example, the trained machine learning model receives input data describing a first sensor 202 insertion site location as well as glucose measurements 118 taken by the sensor 202 in the first insertion site location. The trained machine learning model generates output data describing ideal glucose values at the first sensor 202 insertion site location based on the input data.

In an example, the adaptive system 314 (and/or the computing device 108) computes a difference between each of the glucose values 502-540 and its corresponding ideal glucose value and compares the computed difference to a difference threshold. For example, the adaptive system 314 computes a difference between the glucose value 502 and an ideal glucose value which would have been measured instead of the glucose value 502 if the person 102 had inserted the sensor 202 at an insertion site location on the person's 102 abdomen or buttock instead of on the person's 102 thigh. The adaptive system 314 then compares the difference between the glucose value 502 and the ideal glucose value to the difference threshold. If this difference is less than the difference threshold, then the adaptive system 314 does not modify the glucose data 308 in one example. If the difference is greater than the difference threshold, then the adaptive system 314 (and/or the computing device 108) modifies the glucose data 308 by replacing the glucose value 502 with its corresponding ideal glucose value in another example.

In the illustrated example, the adaptive system 314 determines that differences between each of the glucose values 502-526 and corresponding ideal glucose values are less than the difference threshold. Accordingly, the adaptive system 314 does not modify the glucose values 502-526. For example, the adaptive system 314 determines that differences between each of the glucose values 528-540 and corresponding ideal glucose values are greater than the difference threshold. Based on this determination, the adaptive system 314 replaces the glucose values 528-540 with the glucose values 802-814, respectively, which are the ideal glucose values corresponding to the glucose values 528-540 in this example. As shown, the adaptive system 314 (and/or the computing device 108) generates the modified glucose data by replacing the glucose values 528-540 with the glucose values 802-814.

Consider an example in which the adaptive system 314 leverages the probabilistic model to estimate the effect of the sensor's 202 insertion site location on the person's 102 thigh relative to the glucose values 502-540. In this example, the adaptive system 314 (and/or the computing device 108) determines whether or not to generate the modified glucose data at least partially based on outputs from the probabilistic model. In an example, the adaptive system 314 uses the probabilistic model to generate the ideal glucose values based on the historic heart rate data and the historic glucose data. For example, the adaptive system 314 forms the probabilistic model based on heart rate values described by the historic heart rate data and corresponding glucose values described by the historic glucose data.

Because the probabilistic model is formed in this way, the model outputs a glucose value which is most likely to be observed given an observation of a heart rate value based on the historic heart rate and glucose data. Accordingly, of all of the pairs of heart rate values and glucose values described by the historic heart rate data and the historic glucose data, the probabilistic model identifies a most frequently paired glucose value with a given input heart rate value and the model outputs the identified glucose value. In one example, the adaptive system 314 uses glucose values output by the probabilistic model as the ideal glucose values.

To do so in one example, the adaptive system 314 receives the glucose data 308 and the non-glucose data 310 which includes heart rate data describing measured heart rate values of the person 102. For example, the adaptive system 314 (and/or the computing device 108) identifies a heart rate value having a same timestamp as each of the glucose values 502-540. For each of the glucose values 502-540, the adaptive system 314 determines a corresponding ideal glucose value using the identified heart rate values and the probabilistic model.

Accordingly, for the glucose value 502, the adaptive system 314 first identifies the heart rate value having the same timestamp as the glucose value 502. The adaptive system 314 (and/or the computing device 108) then uses the identified heart rate value as an input to the probabilistic model which receives the input, and then outputs an ideal glucose value corresponding to the glucose value 502. For example, the adaptive system 314 determines a difference between the glucose value 502 and the ideal glucose value and compares this difference to the difference threshold. As shown, the adaptive system 314 determines that the difference is less than the difference threshold, and as a result, the adaptive system 314 does not modify the glucose value 502.

The adaptive system 314 (and/or the computing device 108) determines an ideal glucose value for each of the remaining glucose values 504-540 and compares a difference between each of the glucose values 504-540 and its corresponding ideal glucose value to the difference threshold. As shown, differences between the glucose values 502-526 and corresponding ideal glucose values are less than the difference threshold. However, differences between the glucose values 528-540 and corresponding ideal glucose values are greater than the difference threshold. As a result, the adaptive system 314 (and/or the computing device 108) generates the modified glucose data by replacing the glucose values 528-540 with the glucose values 802-814 which are the ideal glucose values output by the probabilistic model for input heart rate values corresponding to a timestamp of each of the glucose values 528-540.

Consider another example in which the adaptive system 314 leverages the probabilistic model to estimate the effect of the sensor's 202 insertion site location on the person's 102 thigh relative to the glucose values 502-540. In this example, the adaptive system 314 forms the probabilistic model using the historic heart rate data and the historic glucose data such that for an input heart rate value and an input glucose value, the model outputs a probability of observing the input glucose value given an observation of the input heart rate value based on the historic heart rate and glucose data. For example, the adaptive system 314 determines a probability of observing each of the glucose values 502-540 given an observation of a heart rate value which has a same timestamp.

Continuing this example, the adaptive system 314 receives the glucose data 308 and the non-glucose data 310 which includes the heart rate data describing measured heart rate values of the person 102. The adaptive system 314 processes the heart rate data to identify a heart rate value having a same timestamp as each of the glucose values 502-540. For example, the adaptive system 314 inputs the glucose value 502 and a corresponding heart rate value having a same timestamp as the glucose value 502 to the probabilistic model which outputs a probability of observing the glucose value 502 given an observation of the heart rate value that has the same timestamp as the glucose value 502.

The adaptive system 314 (and/or the computing device 108) compares the probability of observing the glucose value 502 with an observance threshold. If the probability is less than the observance threshold, then the adaptive system 314 replaces the glucose value 502 with its corresponding ideal glucose value. If the probability is greater than the observance threshold, then the adaptive system 314 does not replace the glucose value 502 with its corresponding ideal glucose value.

The adaptive system 314 (and/or the computing device 108) repeats this process for each of the glucose values 504-540. As shown in the example depicted in FIG. 8, probabilities of observing the glucose values 502-526 are each greater than the observance threshold and the adaptive system 314 does not replace the glucose values 502-526. As further shown, probabilities of observing the glucose values 528-540 are each less than the observance threshold and the adaptive system 314 replaces each of the glucose values 528-540. For example, the adaptive system 314 (and/or the computing device 108) replaces the glucose values 528-540 with the glucose values 802-814, respectively, which are ideal glucose values that would have been measured or would have likely been measured instead of the glucose values 528-540 if the person 102 was wearing the CGM system 104 such that the location of the sensor's 202 insertion site was on the person's 102 abdomen instead of on the person's 102 thigh.

In some examples, the adaptive system 314 determines the glucose values 802-814 using the sensor 202 insertion site location conversion data. In other examples, the adaptive system 314 determines the glucose values 802-814 using the machine learning model which is trained to generate ideal glucose measurements based on the training data describing the differences between glucose measurements 118 at different sensor 202 insertion site locations on the person 102. For example, the adaptive system 314 may determine the glucose values 802-814 using the probabilistic model in the example in which the probabilistic model is formed based on the heart rate values described by the historic heart rate data and the corresponding glucose values described by the historic glucose data.

In some examples, by replacing the glucose values 528-540 with the glucose values 802-814, the adaptive system 314 improves an accuracy of the CGM system 104 while it is worn with the sensor's 202 insertion site located on the person's 102 thigh. For example, this at least partially mitigates a risk associated with the person 102 wearing the CGM system 104 on the person's 102 thigh which is not an intended location for the CGM system 104 to be worn. In an example in which the sensor's 202 insertion site location of on the person's 102 thigh corresponds to a moderate risk classification, replacing the glucose values 528-540 with the glucose values 802-814 is sufficient to reduce the risk classification from moderate to low.

FIG. 9 illustrates a representation 900 of a user interface for confirming a determined location of a glucose sensor insertion site. As described above, the adaptive system 314 receives the glucose data 308 describing user glucose values for the person 102 and the adaptive system 314 also receives the non-glucose data 310 which includes the orientation data 304 describing forces measured by an accelerometer of the CGM system 104. For example, the adaptive system 314 compares the forces described by the orientation data 304 with characteristic force patterns associated with locations on the person 102 which represent possible sensor 202 insertion site location. Through this comparison, the adaptive system 314 identifies the person's 102 thigh as the location of the sensor's 202 insertion site.

In one example, the adaptive system 314 determines a risk classification for the sensor's 202 insertion site location as being low risk. Accordingly, the adaptive system 314 performs minimal intervention to correct the sensor's 202 insertion site location. As shown, the adaptive system 314 generates the indication 318 for display in a user interface of the computing device 108. In the illustrated example, the indication 318 is a request for the person 102 to confirm that the CGM system 104 is being worn on the person's 102 thigh.

The computing device 108 receives the indication 318 and displays the indication 318 in the user interface of the computing device 108 as “Are you wearing the CGM system on your thigh?” Although the indication 318 does not specifically mention the sensor's 202 insertion site location, if the CGM system 104 is being worn on the person's 102 thigh, then the sensor's 202 insertion site location is the person's 102 thigh. The user interface of the computing device 108 also includes user interface elements 902, 904. For example, the person 102 interacts with the user interface element 902 to indicate that the CGM system 104 is being worn on the person's 102 thigh. Alternatively, the person 102 interacts with the user interface element 904 to indicate that the CGM system 104 is not being worn on the person's 102 thigh.

The computing device 108 transmits data describing the person's 102 response to the indication 318 to the storage device 120 via the network 116. In some examples, the storage device 120 is included in the virtual container 306. For example, the virtual container 306 limits access to the data describing the person's 102 response. In one example, the indication 318 also informs the person 102 that access to the data describing the person's 102 response will be limited by the virtual container 306. In this manner, the person 102 is more likely to interact with the user interface element 902 even if the person 102 is aware that wearing the CGM system 104 on the person's 102 thigh is not an indicated location for wearing the CGM system 104.

The adaptive system 314 receives the data describing the person's 102 response. For example, the data describing the person's 102 response is included in the non-glucose data 310 and the adaptive system 314 processes the data describing the person's 102 response to determine whether or not to modify the glucose data 308. In an example in which the data describing the person's 102 response describes an interaction with the user interface element 904, the adaptive system 314 may not modify the glucose data 308. In this example, the adaptive system 314 generates an additional indication 318 for display in the user interface of the computing device 108 which is a prompt for the person 102 to indicate a worn location of the CGM system 104. This indicated worn location of the CGM system 104 corresponds to the sensor's 202 insertion site.

In an example in which the data describing the person's 102 response describes an interaction with the user interface element 902, the adaptive system 314 can modify the glucose data 308 as previously described. In this example, the adaptive system 314 generates the modified data 316 by modifying the glucose data 308 based on the location of the sensor's 202 insertion site on the person's 102 thigh. For example, the adaptive system 314 generates an additional indication 318 for display in the user interface of the computing device 108 which indicates how the glucose data 308 was modified based on the location of the sensor's 202 insertion site.

In another example, the adaptive system 314 modifies the glucose data 308 in a manner that is not necessarily communicated to the person 102. In some examples, the adaptive system 314 determines whether to generate the additional indication 318 (e.g., which indicates how the glucose data 308 was modified) based on a difference between the glucose data 308 and the modified data 316. If this difference is relatively small, then the person 102 may consider the additional indication 318 to be a nuisance. Accordingly, the adaptive system 314 may not generate the additional indication 318 in response to determining that the difference between the glucose data 308 and the modified data 316 is relatively small.

Consider another example in which the adaptive system 314 determines not to inform the person 102 with respect to how glucose data 308 is modified. In this example, the adaptive system 314 determines that the difference between the glucose data 308 and the modified data 316 does not correspond to a scenario in which an action or intervention by the person 102 would be beneficial. For example, the adaptive system 314 does not inform the person 102 with respect to how the glucose data 308 is modified because there is nothing beneficial for the person 102 to do with this information. In one example, the adaptive system 314 does not inform the person 102 with respect to how the glucose data 308 is modified to avoid a risk of the person 102 acting or intervening based on a belief that such an action or intervention is necessary.

In some examples in which the adaptive system 314 determines not to inform the person 102 with respect to how glucose data 308 is modified, the adaptive system 314 instead generates the additional indication 318 for the person's 102 healthcare provider. In these examples, the adaptive system 314 communicates the additional indication 318 to a computing device of the healthcare provider. In this manner, computing device of the healthcare provider displays the additional indication 318 in a user interface for the healthcare provider. The healthcare provider communicates a significance of the modification of the glucose data 308 to the person 102. Accordingly, the adaptive system 314 avoids communicating information to the person 102 which the person 102 perceives as a nuisance.

If the adaptive system 314 determines that the difference between the glucose data 308 and the modified data 316 is large or otherwise significant, then the adaptive system 314 generates the additional indication 318 that indicates how the glucose data 308 was modified, and the computing device 108 displays the additional indication 318 for the person 102. For example, the adaptive system 314 generates the additional indication 318 based on determining that the difference between the glucose data 308 and the modified data 316 corresponds to a scenario in which action or intervention by the person 102 would be beneficial. In some examples, the action or the intervention by the person 102 is a current action or intervention. In other examples, the action or the intervention by the person 102 is a future action or intervention.

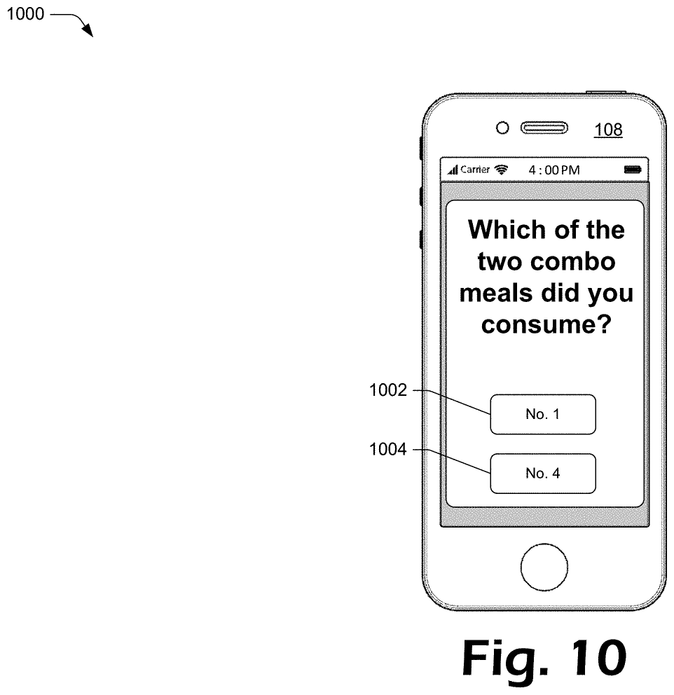

FIG. 10 illustrates a representation 1000 of a user interface for identifying which meal of multiple purchased meals was consumed by a user of a continuous glucose monitoring (CGM) system. For example, the user of the CGM system 104 is the person 102 and the adaptive system 314 (and/or the computing device 108) monitors carbohydrates consumed by the person 102. To do so in one example, the adaptive system 314 leverages consumption data describing food consumed by the person 102 and acquisition data describing food acquired by the person 102. In this example, the non-glucose data 310 includes the consumption data and the acquisition data.

In some examples, the person 102 generates the consumption data by interacting with a user interface of the computing device 108 to indicate food (e.g., meals, snacks, supplements, etc.) that the person 102 has consumed. In one example, the computing device 108 receives the acquisition data via the IoT 114. For example, the adaptive system 314 receives the non-glucose data 310 which includes the consumption data and the acquisition data. The adaptive system 314 (and/or the computing device 108) processes the consumption data and the acquisition data to monitor carbohydrates consumed by the person 102 in relation to the glucose measurements 118.

To do so in one example, the adaptive system 314 (and/or the computing device 108) cross-references acquired food described by the acquisition data with consumed food described by the consumption data. For example, the acquisition data describes various types of food acquired by the person 102 such as purchases at grocery stores and purchases at restaurants. The adaptive system 314 (and/or the computing device 108) identifies food described by the acquisition data which is likely to be consumed by the person 102.

In one example, the adaptive system 314 determines that food acquired via a purchase at a restaurant is more likely to be consumed by the person 102 than food acquired via a purchase at a grocery store. In another example, the adaptive system 314 (and/or the computing device 108) can infer a time period within which the food acquired via the purchase at the restaurant will likely be consumed by the person 102. In some examples, the acquisition data describes digital images depicting the food (e.g., captured via an image capture device of the computing device 108). In these examples, the digital images are processed by a machine learning model of the computing device 108 and/or the adaptive system 314 to determine which acquired food is likely to be consumed by the person 102. For example, the machine learning model is trained on training data describing first sets of digital images depicting food which is consumed by a person that acquired the food and second sets of digital images depicting food which is not consumed by a person that acquired the food.

Regardless of a manner in which the adaptive system 314 (and/or the computing device 108) identifies food described by the acquisition data which is likely to be consumed by the person 102, these identifications are comparable to consumed food described by the consumption data. In one example, the adaptive system 314 cross-references the identified food which is likely to be consumed by the person 102 with consumed food described by the consumption data that was consumed by the person 102. For example, if the adaptive system 314 (and/or the computing device 108) determines that particular food identified as likely to be consumed is currently described by the consumption data as consumed food, then the adaptive system 314 continues to process the consumption data and the acquisition data to monitor carbohydrates consumed by the person 102.

If the adaptive system 314 determines that the particular food identified as likely to be consumed is not described by the consumption data as consumed food (e.g., within a threshold time period following acquisition of the particular food), then the adaptive system 314 (and/or the computing device 108) processes the consumption data to identify gaps. For example, a gap in the consumption data is food consumed by the person 102 but not recorded or generated as consumption data by the person 102 interacting with the user interface of the computing device 108. In one example, the adaptive system 314 (and/or the computing device 108) determines that the consumption data describes a first day of consumed food including two meals (e.g., a breakfast and a lunch) and a next day of consumed food including three meals (e.g., a breakfast, a lunch, and a dinner). In this example, the adaptive system 314 identifies a gap in the consumption data as a third meal on the first day which was likely consumed by the person 102 but not recorded or generated as consumption data by the person 102.

Continuing the previous example, the adaptive system 314 (and/or the computing device 108) determines whether the gap in the consumption data corresponds to the particular food identified as likely to be consumed which is not described by the consumption data as consumed food. For example, adaptive system 314 compares a timestamp corresponding to an acquisition of the particular food identified as likely to be consumed with an approximate time of the third meal on the first day. If the adaptive system 314 (and/or the computing device 108) determines that the gap in the consumption data corresponds to the particular food identified as likely to be consumed that is not described by the consumption data as consumed food, then the adaptive system 314 may generate the indication 318 as a request for conformation that the particular food identified as likely to be consumed was consumed by the person 102 as the third meal on the first day. In this example, the computing device 108 receives the indication 318 and renders the request for conformation in the user interface of the computing device 108.

Consider an example in which the adaptive system 314 (and/or the computing device 108) generates the indication 318 to clarify additional information as part of monitoring carbohydrates consumed by the person 102. In this example, the acquisition data describes food acquired from a fast-food restaurant. In particular, the acquisition data describes that two combo meals are acquired by the person 102 from the fast-food restaurant. The adaptive system 314 (and/or the computing device 108) processes the acquisition data and determines that it is unlikely that the person 102 consumed both of the combo meals. In response to this determination, the adaptive system 314 generates the indication 318. The computing device 108 receives the indication 318 and displays the indication 318 in the user interface of the computing device 108.

As shown in FIG. 10, the indication 318 is a clarification request of “which of the two combo meals did you consume?” in this example. For example, the user interface of the computing device 108 includes user interface elements 1002, 1004. The person 102 interacts with user interface element 1002 to indicate that the person 102 consumed a “no. 1” and/or the person 102 interacts with user interface element 1004 to indicate that the person 102 consumed a “no. 4.” In an example in which the person 102 interacts with both of the user interface elements 1002, 1004, the adaptive system 314 classifies both of the combo meals as food acquired and likely consumed by the person 102.

The adaptive system 314 (and/or the computing device 108) leverages the consumption data to support a variety of different functionalities such as estimating the person's 102 carbohydrate consumption and using the estimated carbohydrate consumption to predict the person's 102 future glucose levels. If the person's 102 predicted future glucose levels are greater than a high threshold or lower than a low threshold, then the adaptive system 314 can generate the indication 318 as an alert which provides an opportunity for the person 102 to increase the person's 102 time in range (TIR). In one example, the adaptive system 314 uses the person's 102 estimated carbohydrate consumption to identify relationships between the person's 102 blood glucose levels and consumption of carbohydrates which can differ between the person 102 and the user population 110.

For example, the person's 102 blood glucose level response to consumption of carbohydrates may not be shared by another person in the user population 110. In other examples, the adaptive system 314 leverages the person's 102 estimated carbohydrate consumption when forming the probabilistic model such as to improve an accuracy of the model by correlating observed user glucose values and carbohydrate consumption events. In an example, the adaptive system 314 (and/or the computing device 108) uses the person's 102 estimated carbohydrate consumption as part of decision support in meal and exercise planning for the person 102 to maximize the person's 102 TIR.

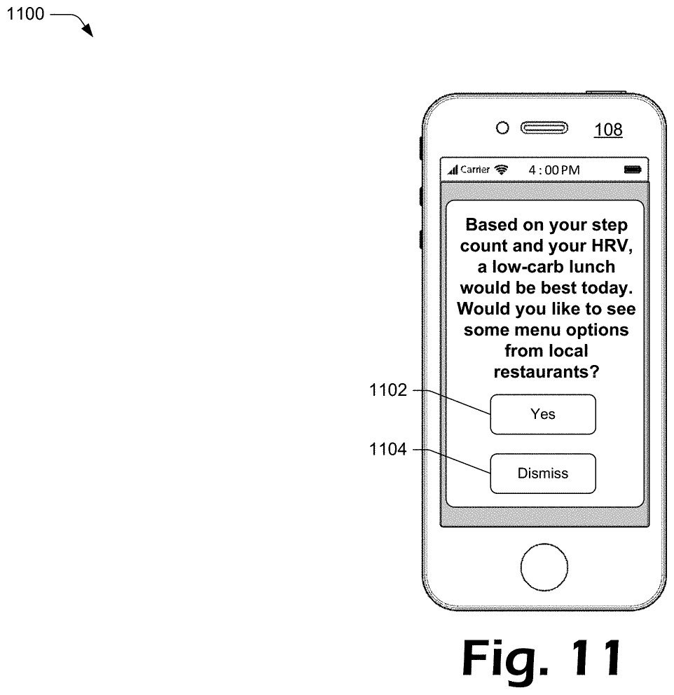

FIG. 11 illustrates a representation 1100 of a user interface for decision support in meal planning. In the representation 1100, the CGM system 104 includes an accelerometer and a heart rate monitor, for example, the additional sensors 220 include the accelerometer and the heart rate monitor. The accelerometer measures forces caused by movements of the person 102 and the sensor module 204 receives communications from the accelerometer describing the measured forces. The sensor module 204 processes these communications from the accelerometer to generate step data describing steps taken by the person 102. For example, the computing device 108 receives CGM device data 214 that includes the step data describing the steps taken by the person 102.

The heart rate monitor measures changes in blood volume corresponding to beats of the person's 102 heart. The sensor module 204 receives communications from the heart rate monitor describing the changes in blood volume corresponding to beats of the person's 102 heart. In one example, the sensor module 204 generates heart rate data describing the changes in blood volume corresponding to beats of the person's 102 heart. In this example, the computing device 108 receives CGM device data 214 that includes the heart rate data describing the changes in blood volume corresponding to beats of the person's 102 heart.

Consider an example in which the adaptive system 314 uses the person's 102 estimated carbohydrate consumption as described above along with the steps data and the heart rate data to form a meal planning model which can be a probabilistic model, a trained machine learning model, and so forth. For example, the virtual container 306 limits access to historic carbohydrate data describing the person's 102 historic estimated carbohydrate consumption, historic steps data describing the person's 102 historic steps taken, historic heart rate data describing historic measured heart rate values of the person 102, and/or historic glucose data describing the person's 102 historic glucose values. In an example in which the meal planning model is implemented as a probabilistic model, the adaptive system 314 forms the meal planning model as three separate probabilistic models.

Continuing the previous example, a first probabilistic model is formed based on the historic carbohydrate data and the historic glucose data such that the first probabilistic model receives a carbohydrate consumption value and a user glucose value as an input and the first probabilistic model outputs a probability of observing the user glucose value given an observation of the carbohydrate consumption value based on the historic data. A second probabilistic model is formed based on the historic heart rate data and the historic glucose data such that the second probabilistic model receives a heart rate variability value and a user glucose value as an input and the second probabilistic model outputs a probability of observing the user glucose value given an observation of the heart rate variability value based on the historic data. A third probabilistic model is formed based on the historic steps data and the historic glucose data such that the third probabilistic model receives a step count value and a user glucose value as an input and the third probabilistic model outputs a probability of observing the user glucose value given an observation of the step count value based on the historic data.

In an example in which the meal planning model is implemented as a machine learning model, the historic carbohydrate data, the historic steps data, the historic heart rate data, and/or the historic glucose data is leveraged as training data for training the machine learning model. By using instances of observed carbohydrate consumption values, observed step count values, observed heart rate variability values, and observed user glucose values as training data, the machine learning model learns to predict a user glucose value given an observed carbohydrate consumption value, an observed step count value, and/or an observed heart rate variability value. In some examples, the training data includes pairs of observed carbohydrate consumption values and corresponding observed user glucose values; observed step count values and corresponding observed user glucose values; and observed heart rate variability values and corresponding observed user glucose values.

As shown in FIG. 11, the adaptive system 314 (and/or the computing device 108) leverages the meal planning model for decision support in meal planning. For example, using the meal planning model, the adaptive system 314 determines that consuming a minimal amount of carbohydrates at the person's 102 next meal will increase a probability of increasing the person's 102 TIR. Based on this determination, the adaptive system 314 generates the indication 318 to communicate that the person 102 should avoid a next meal which is high in carbohydrates. As shown, the computing device 108 receives the indication 318 which is displayed in the user interface of the computing device as “based on your step count and your HRV, a low-carb lunch would be best today. Would you like to see some menu options from local restaurants?” The user interface also includes user interface elements 1102, 1104. The person 102 interacts with user interface element 1102 to see menu options or the person 102 interacts with user interface element 1104 to dismiss the indication 318.

Consider an example in which the adaptive system 314 uses the historic carbohydrate data, the historic steps data, the historic heart rate data, and/or the historic glucose data to reduce a number of nuisance alerts or alarms generated and/or displayed for the person 102. In this example, the adaptive system 314 (and/or the computing device 108) processes the glucose data 308 using a temporal window that ends at a time corresponding to a timestamp of a most recent user glucose value described by the glucose data 308. The adaptive system 314 generates the indication 318 as an alarm if the most recent user glucose value described by the glucose data 308 is above a high glucose level threshold or below a low glucose level threshold. The adaptive system 314 generates the indication 318 as an alert if a trend in the user glucose values described by the glucose data 308 indicates that the person's 102 glucose levels will be too high soon or too low soon.

However, in some examples, the adaptive system 314 generates the indication 318 as an alert based on normal fluctuations of the person's 102 glucose levels which appear as a false positive trend that the person's 102 glucose levels will be too high or too low soon. In these examples, the indication 318 is a nuisance alert. For example, the adaptive system 314 uses the historic carbohydrate data, the historic steps data, the historic heart rate data, and/or the historic glucose data to reduce a likelihood of generating a nuisance alert.

To do so, the adaptive system 314 (and/or the computing device 108) first identifies a trend in the user glucose values described by the glucose data 308 which indicates that the person's 102 glucose levels will be too high soon or too low soon. Before generating the indication 318 as an alert based on the identified trend in the glucose data 308, the adaptive system 314 identifies at least one supporting trend from the historic carbohydrate data, the historic steps data, and/or the historic heart rate data that also indicates that the person's 102 glucose levels will be too high soon or too low soon. For example, if the adaptive system 314 (and/or the computing device 108) identifies the trend in the user glucose values described by the glucose data 308 and if the adaptive system 314 identifies the at least one supporting trend from the historic carbohydrate data, the historic steps data, and/or the historic heart rate data, then the adaptive system 314 generates the indication 318 as the alert. Alternatively, if the adaptive system 314 identifies the trend in the user glucose values described by the glucose data 308 and if the adaptive system 314 does not identify the at least one supporting trend, then the adaptive system 314 does not generate the indication 318 as the alert. By leveraging the at least one supporting trend in this manner, the adaptive system 314 significantly reduces a number of nuisance alerts generated and displayed for the person 102.

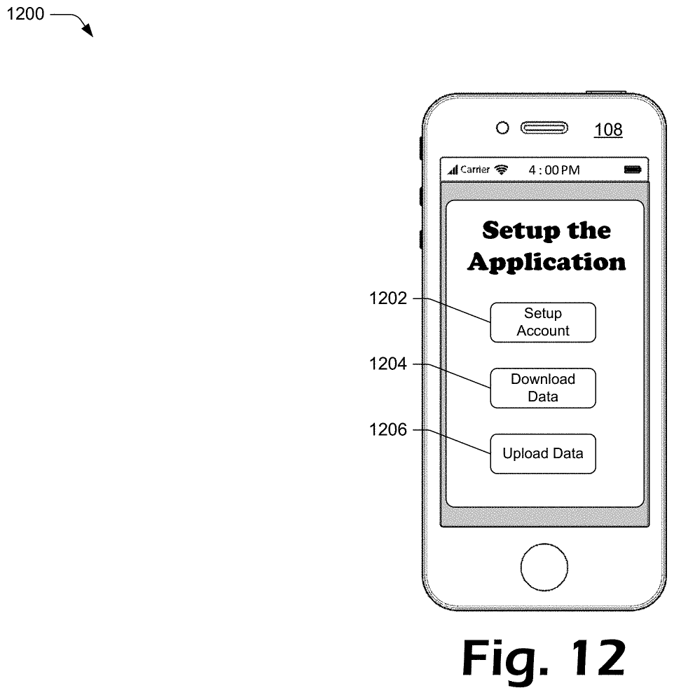

FIG. 12 illustrates a representation 1200 of a user interface for setting up a continuous glucose monitoring (CGM) system. In one example, the computing device 108 changes a display in the user interface based on a source of the CGM device data 214. For example, the CGM device data 214 is from a source that indicates the person 102 should setup a new application for monitoring the person's 102 glucose values. As shown, the computing device 108 displays user interface elements 1202, 1204, 1206 based on the source of the CGM device data 214. In an example, the person 102 interacts with user interface element 1202 to setup an account. For example, the person 102 interacts with user interface element 1204 to download data. In one example, the person 102 interacts with user interface element 1206 to upload data.

### Asynchronous Display Rates

For example, the computing device 108 changes a display rate for the user interface based on a source of the CGM device data 214. In some examples, the display rate for the user interface is asynchronous while in other examples the display rate for the user interface is synchronous based on the source of the CGM device data 214. In an example, the CGM system 104 transmits the CGM device data 214 to the computing device 108 every 30 seconds and the computing device 108 uses the source of the CGM device data 214 and a transmission rate of the CGM device data 214 to change the display rate for the user interface.

Consider an example in which the computing device 108 modifies a display rate for displaying the glucose measurements 118 based on a device type of the computing device 108 to minimize power consumption by the computing device 108. For example, the computing device 108 displays the glucose measurements 118 asynchronously to minimize power consumption by the computing device 108. In this example, the CGM system 104 transmits the CGM device data 214 to the computing device 108 every 30 seconds. In an example in which the computing device 108 is a smartphone, the computing device 108 displays the glucose measurements 118 every minute to maximize a battery life of the computing device 108. In an example in which the computing device 108 is a smart watch, the computing device 108 displays the glucose measurements 118 every five minutes to maximize a battery life of the computing device 108.

For example, the computing device 108 reduces a display rate for the glucose measurements 118 if the computing device 108 is a low resource device and the computing device 108 increases a display rate for the glucose measurements 118 if the computing device 108 is not a low resource device. In some examples, the computing device 108 changes a display rate for the glucose measurements 118 based on a classification of the person 102. In one example, if the person 102 is a premium user as part of a paid subscription, then the computing device 108 displays the glucose measurements 118 every 30 seconds as they are received from the CGM system 104. If the person 102 is not a premium user as part of the paid subscription, then the computing device 108 displays the glucose measurements 118 every two minutes.

Consider an example in which the computing device 108 changes a display rate for the glucose measurements 118 based on whether or not the person 102 has Type 1 or Type 2 diabetes. In this example, if the person 102 has Type 1 diabetes, then the computing device 108 displays the glucose measurements 118 every 30 seconds as they are received from the CGM system 104. If the person 102 has Type 2 diabetes, then the computing device 108 displays the glucose measurements 118 every minute. For example, if the person 102 does not have Type 1 or Type 2 diabetes, then the computing device 108 displays the glucose measurements 118 every five minutes.

In some examples, the computing device 108 changes a display rate for the glucose measurements 118 based on a remaining amount of electrical charge of a power supply (e.g., a battery) which supplies power to the computing device 108. In one example, if the remaining amount of electrical charge of the power supply is greater than a first charge threshold (e.g., 50 percent), then the computing device 108 displays the glucose measurements 118 every 30 seconds as the computing device 108 receives the CGM device data 214. For example, if the remaining amount of electrical charge of the power supply is below the first charge threshold and above a second charge threshold (e.g., 10 percent), then the computing device 108 displays the glucose measurements 118 every minute. If the remaining amount of electrical charge of the power supply is below the second charge threshold, then the computing device 108 displays the glucose measurements 118 every five minutes in one example.

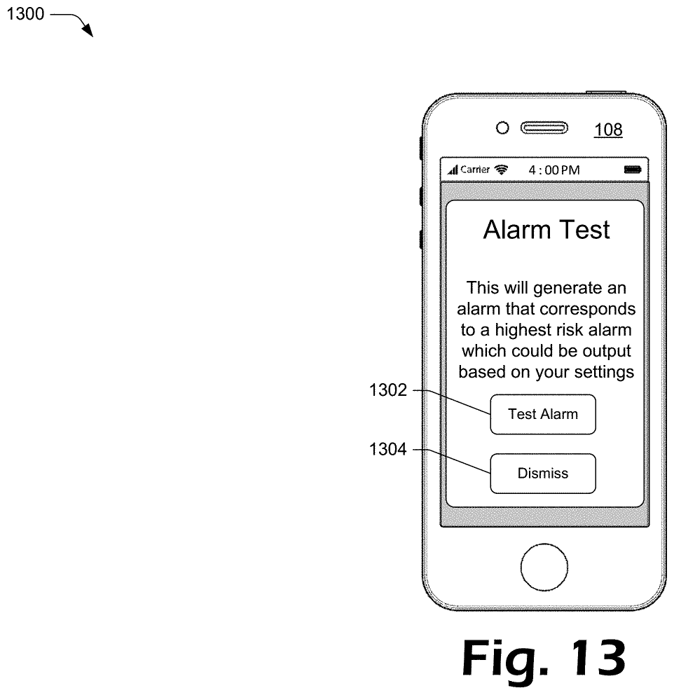

FIG. 13 illustrates a representation 1300 of a user interface for testing alarms of a continuous glucose monitoring (CGM) system. In some examples, the computing device 108 is a medical device as defined by the United States Food and Drug Administration (USFDA). In these examples, the computing device 108 is subject to medical device regulations and requirements. In one example in which the computing device 108 is a medical device, the computing device 108 is subject to pre-market clearance or approval, medical device design and manufacturing standards, medical device reporting standards, and so forth.

For example, if the computing device 108 is a medical device, then medical device directives and international standards specify requirements for alarms generated by the computing device 108. Examples of such requirements include volume requirements, readability requirements, duration requirements, etc. In an example, a user of the computing device 108 is prevented from adjusting settings for alarms generated by the computing device 108. In this example, the user of the computing device 108 may not be able to reduce a volume for an alarm below a particular volume level when the computing device 108 is a medical device.

In other examples, the computing device 108 is not a medical device as defined by the USFDA. In these examples, the computing device 108 may receive data from a medical device (e.g., the CGM system 104) without being defined as a medical device. In an example in which the computing device 108 is not a medical device, the computing device 108 is not subject to requirements for alarms generated by a medical device. In one example, a user of the computing device 108 is able to adjust settings for alarms generated by the computing device 108.

### Alarm Testing

As shown in the representation 1300, the user interface of the computing device 108 is displaying an alarm test interface. The alarm test interface displays “this will generate an alarm that corresponds to a highest risk alarm which could be output based on your settings.” The user interface also includes user interface elements 1302, 1304. For example, regardless of whether the computing device 108 is a medical device or is not a medical device, the person 102 interacts with user interface element 1302 to generate a highest risk alarm (e.g., loudest, longest, brightest, etc.). This allows the person 102 to view and/or hear the highest risk alarm which prevents unnecessary anxiety for the person 102 in an event that the adaptive system 314 generates the indication 318 as the highest risk alarm.

For example, the person 102 interacts with user interface element 1304 to dismiss the alarm test interface. In one example, and in response to the person 102 interacting with the user interface element 1304, the computing device 108 displays an indication of settings for the alarm which are adjustable by the person 102. By allowing the person 102 to experience the highest risk alarm regardless of whether the computing device 108 is a medical device, the person 102 understands what to expect in the event that the adaptive system 314 generates the indication 318 as the highest risk alarm. In some examples, this avoids confusing and/or startling the person 102 in the event that the adaptive system 314 generates the indication 318 as the highest risk alarm and the person 102 has not seen and/or heard the highest risk alarm previously.

Example Procedures

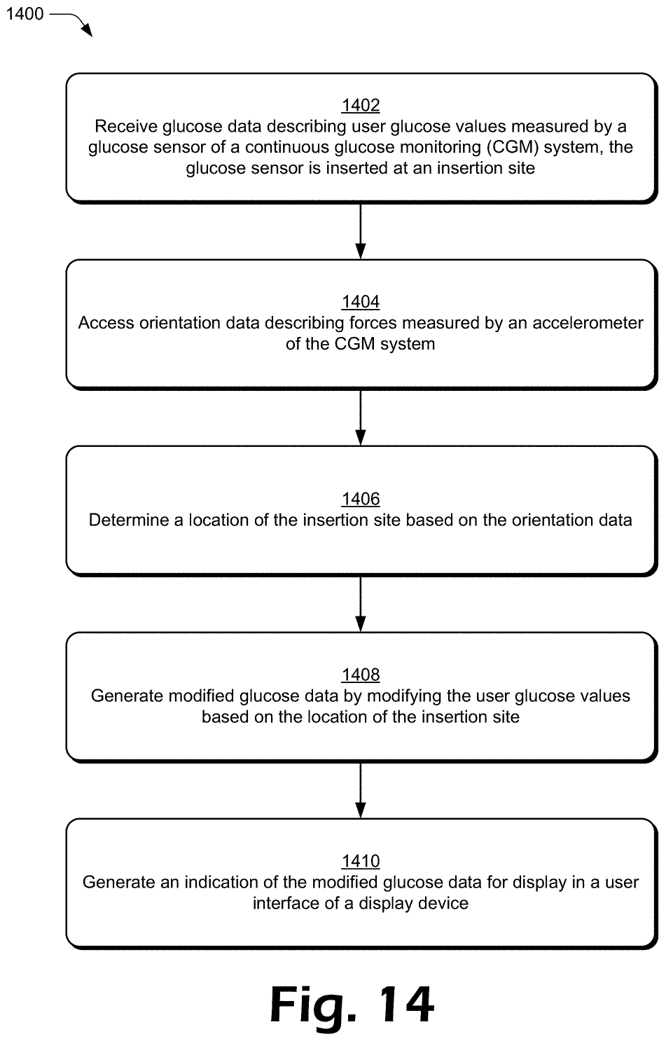

This section describes example procedures for determining similarity of sequences of glucose values. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. FIG. 14 is a flow diagram depicting a procedure 1400 in an example implementation in which glucose data describing user glucose values is received, modified glucose data is generated based on a location of an insertion site of a glucose sensor, and an indication of the modified glucose data is generated for display in a user interface. Glucose data is received describing user glucose values measured by a glucose sensor of a continuous glucose monitoring (CGM) system (block 1402), the glucose sensor is inserted at an insertion site. For example, the adaptive system 314 receives the glucose data 308 describing the user glucose values measured by a glucose sensor of the CGM system 104.

Orientation data is accessed describing forces measured by an accelerometer of the CGM system (block 1404). In one example, the adaptive system 314 accesses the orientation data 304 included in the non-glucose data 310. A location of the insertion site is determined based on the orientation data (block 1406). The adaptive system 314 determines the location of the insertion site based on the orientation data 304 in some examples. Modified glucose data is generated by modifying the user glucose values based on the location of the insertion site (block 1408). For example, the adaptive system 314 generates the modified glucose data based on the location of the insertion site. An indication is generated of the modified glucose data for display in a user interface of a display device (block 1410). In an example, the adaptive system 314 generates the indication of the modified glucose data.

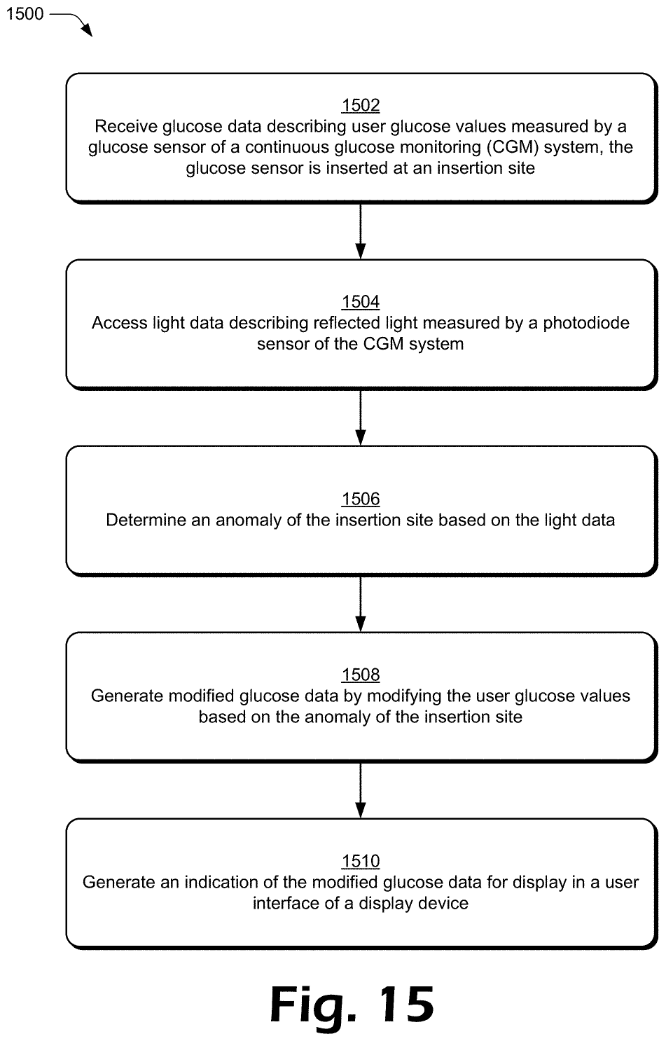

FIG. 15 is a flow diagram depicting a procedure 1500 in an example implementation in which glucose data describing user glucose values is received, modified glucose data is generated based an anomaly of an insertion site of a glucose sensor, and an indication of the modified glucose data is generated for display in a user interface. Glucose data is received describing user glucose values measured by a glucose sensor of a continuous glucose monitoring (CGM) system (block 1502), the glucose sensor is inserted at an insertion site. For example, the adaptive system 314 receives the glucose data 308 describing the user glucose values measured by the glucose sensor of the CGM system 104. Light data is accessed describing reflected light measured by a photodiode of the CGM system (block 1504). The adaptive system 314 accesses the light data in some examples.

An anomaly of the insertion site is determined based on the light data (block 1506). For example, the adaptive system 314 determines the anomaly of the insertion site based on the light data. Modified glucose data is generated by modifying the user glucose values based on the anomaly of the insertion site (block 1508). In an example, the adaptive system 314 generates the modified glucose data based on the anomaly of the insertion site. An indication of the modified glucose data is generated for display in a user interface of a display device (block 1510). In one example, the adaptive system 314 generates the indication of the modified glucose data.

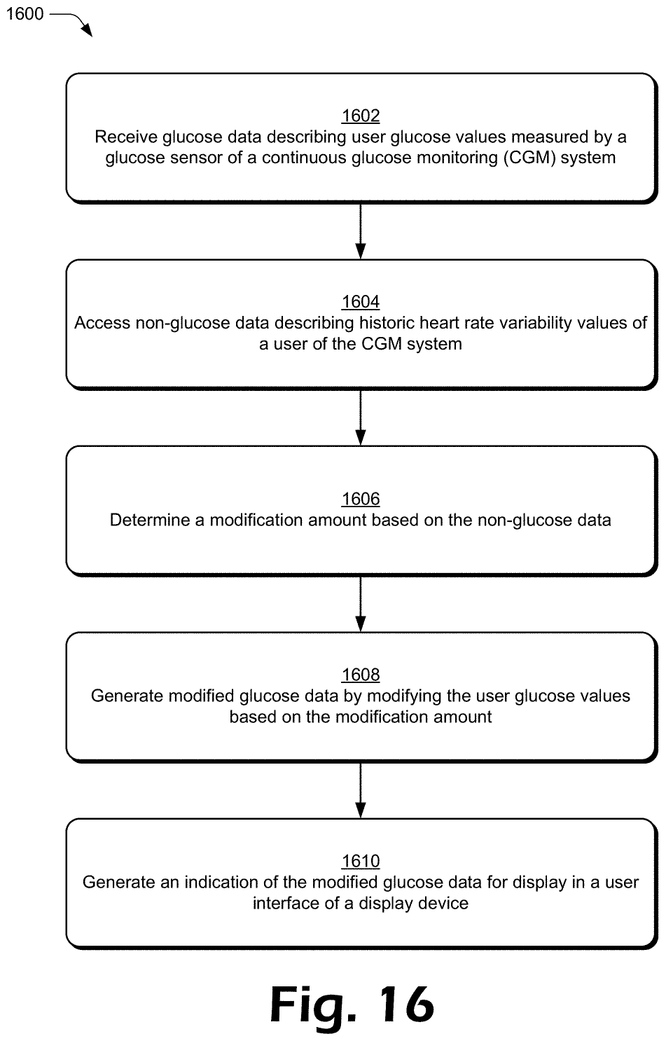

FIG. 16 is a flow diagram depicting a procedure 1600 in an example implementation in which glucose data describing user glucose values is received, a modification amount is determined based on non-glucose data, and modified glucose data is generated by modifying the user glucose values based on the modification amount. Glucose data is received describing user glucose values measured by a glucose sensor of a continuous glucose monitoring (CGM) system (block 1602). In one example, the adaptive system 314 receives the glucose data 308 describing the user glucose values. Non-glucose data is accessed describing historic heart rate variability values of a user of the CGM system (block 1604). For example, the adaptive system 314 accesses the non-glucose data 310.

A modification amount is determined based on the non-glucose data (block 1606). In one example, the adaptive system 314 determines the modification amount. Modified glucose data is generated by modifying the user glucose values based on the modification amount (block 1608). The adaptive system 314 generates the modified glucose data in one example. An indication of the modified glucose data is generated for display in a user interface of a display device (block 1610). For example, the adaptive system 314 generates the indication of the modified glucose data.

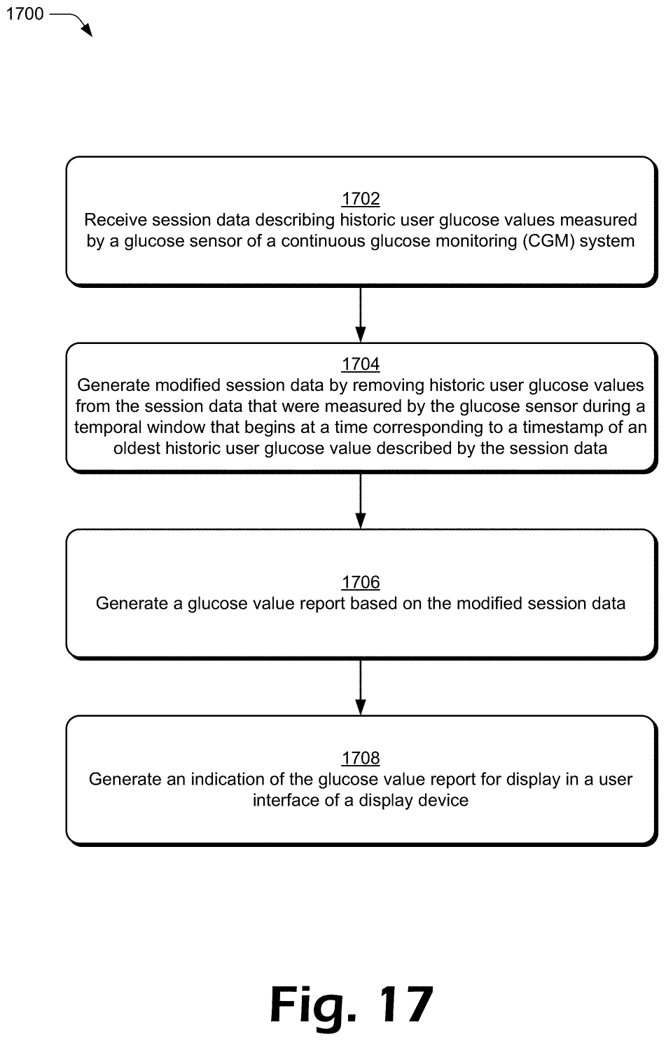

FIG. 17 is a flow diagram depicting a procedure 1700 in an example implementation in which session data describing historic user glucose values is received, modified session data is generated by removing historic user glucose values from the session data that were measured by a glucose sensor during a temporal window, and a glucose value report is generated based on the modified session data. Historic session data is received describing historic user glucose values measured by a glucose sensor of a continuous glucose monitoring (CGM) system (block 1702). For example, the adaptive system 314 receives the historic session data.

Modified session data is generated by removing historic user glucose values from the session data that were measured by the glucose sensor during a temporal window that begins at a time corresponding to a timestamp of an oldest historic user glucose value described by the session data (block 1704). The adaptive system 314 generates the modified session data in one example. A glucose value report is generated based on the modified session data (block 1706). For example, the adaptive system 314 generates the glucose value report based on the modified session data. An indication of the glucose value report is generated for display in a user interface of a display device (1708). In some examples, the adaptive system 314 generates the indication of the glucose value report.

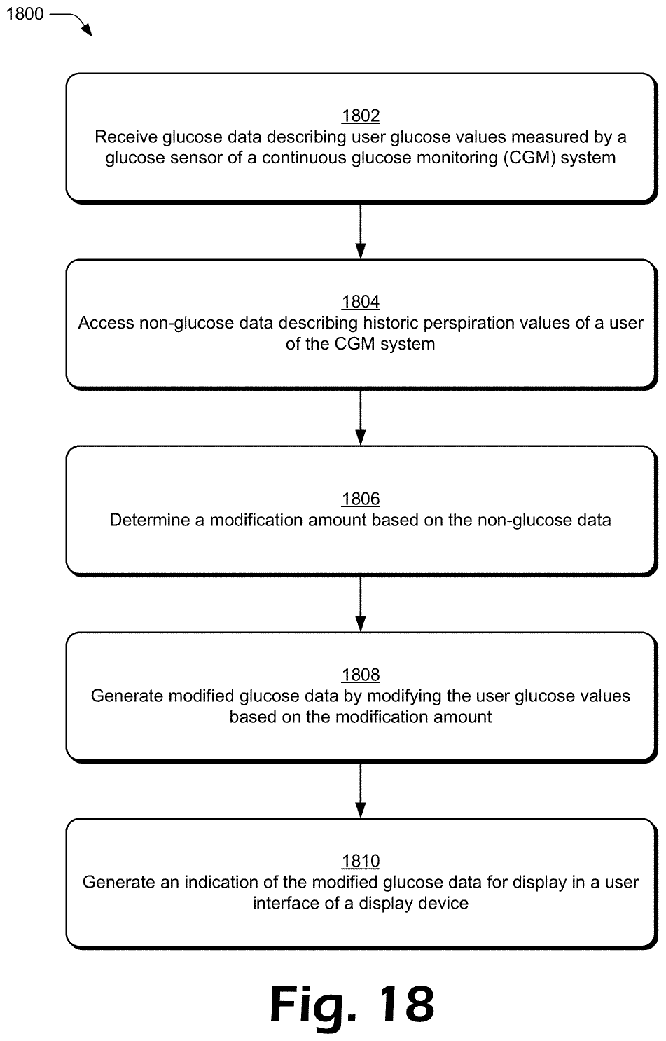

FIG. 18 is a flow diagram depicting a procedure 1800 in an example implementation in which glucose data describing user glucose values is received, a modification amount is determined based on non-glucose data describing historic perspiration values of a user of the CGM system, and modified glucose is generated by modifying the user glucose values based on the modification amount. Glucose data is received describing user glucose values measured by a glucose sensor of a continuous glucose monitoring (CGM) system (block 1802). For example, the adaptive system 314 receives the glucose data. Non-glucose data is accessed that describes historic perspiration values of a user of the CGM system (block 1804). In an example, the adaptive system 314 accesses the non-glucose data.

A modification amount is determined based on the non-glucose data (block 1806). The adaptive system 314 determines the modification amount in some examples. Modified glucose data is generated by modifying the user glucoses values based on the modification amount (block 1808). In some examples, the adaptive system 314 generates the modified glucose data. An indication of the modified glucose data is generated for display in a user interface of a display device (block 1810). For example, the adaptive system 314 generates the indication of the modified glucose data.

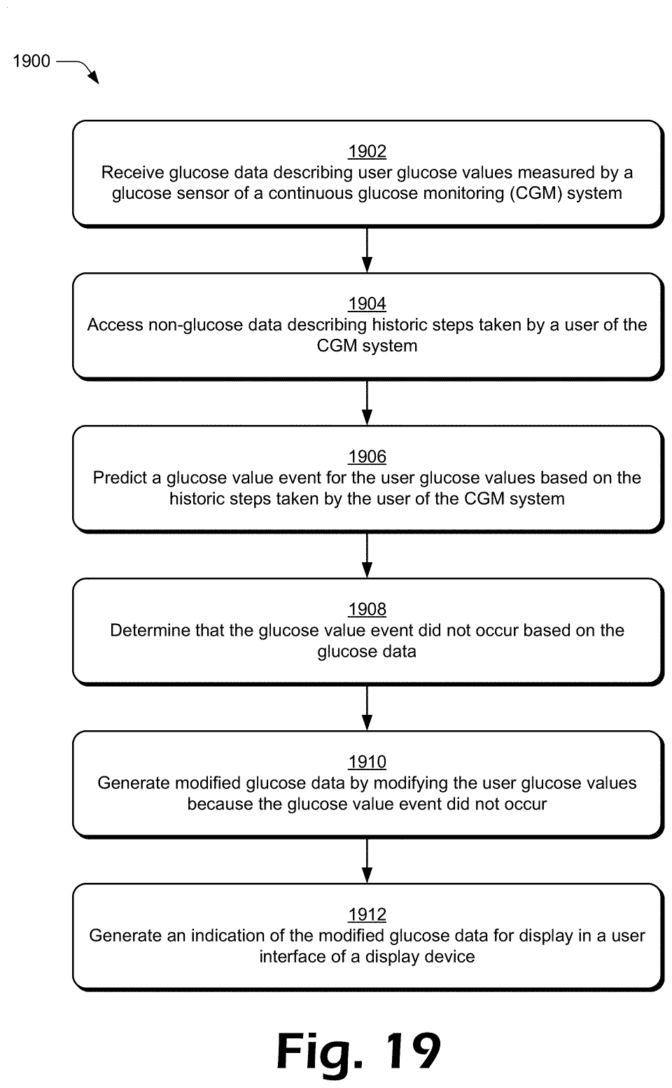

FIG. 19 is a flow diagram depicting a procedure 1900 in an example implementation in which glucose data describing user glucose values is received, a glucose value event is predicted, and modified glucose data is generated because the glucose value event did not occur. Glucose data describing user glucose values measured by a glucose sensor of a continuous glucose monitoring (CGM) system is received (block 1902). The adaptive system 314 receives the glucose data in one example. Non-glucose data is accessed describing historic steps taken by a user of the CGM system (block 1904). For example, the adaptive system 314 accesses the non-glucose data describing the historic steps taken by the user of the CGM system.

A glucose value event is predicted for the user glucose values based on the historic steps taken by the user of the CGM system (block 1906). In one example, the adaptive system 314 predicts the glucose value event for the user glucose values. It is determined that the glucose value event did not occur based on the glucose data (block 1908). The adaptive system 314 determines that the glucose value event did not occur based on the glucose data in an example. Modified glucose data is generated by modifying the user glucose values because the glucose value event did not occur (block 1910). In an example, the adaptive system 314 generates the modified glucose data. An indication of the modified glucose data is generated for display in a user interface of a display device (block 1912). For example, the adaptive system 314 generates the indication of the modified glucose data.

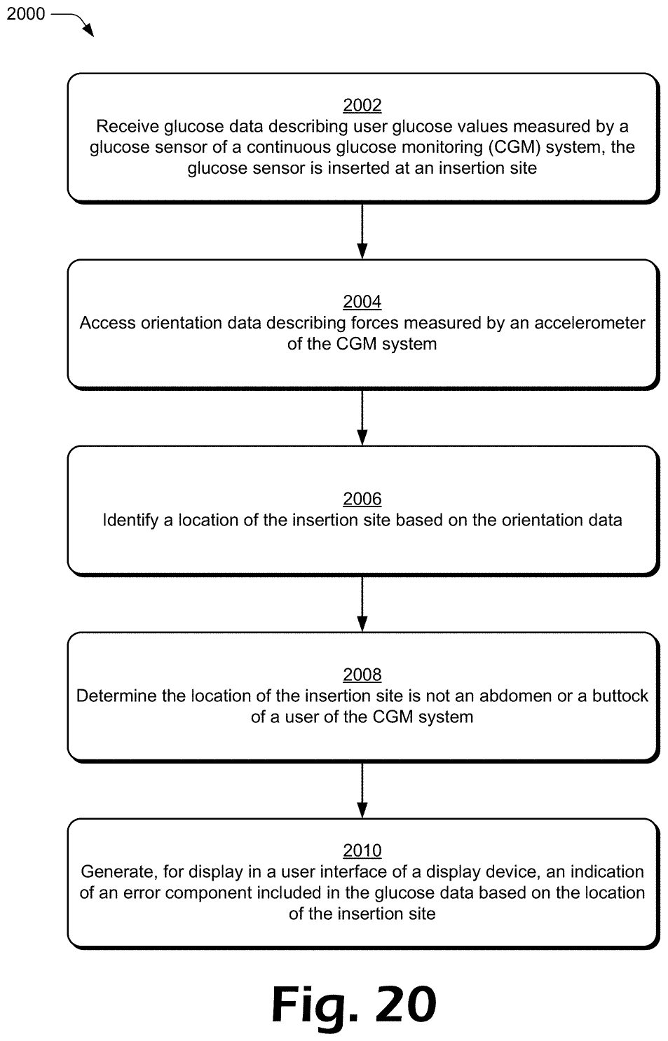

FIG. 20 is a flow diagram depicting a procedure 2000 in an example implementation in which glucose data describing user glucose values is received, a location of an insertion site of a glucose sensor is identified, and an indication of an error component included in the glucose data is generated for display in a user interface based on the location of the insertion site. Glucose data is received describing user glucose values measured by a glucose sensor of a continuous glucose monitoring (CGM) system, the glucose sensor is inserted at an insertion site (block 2002). In an example, the adaptive system 314 receives the glucose data. Orientation data is accessed describing forces measured by an accelerometer of the CGM system (block 2004). For example, the adaptive system 314 accesses the orientation data.

A location of the insertion site is identified based on the orientation data (block 2006). The adaptive system 314 identifies the location of the insertion site in one example. It is determined that the location of the insertion site is not an abdomen or a buttock of a user of the CGM system (block 2008). In one example, the adaptive system 314 determines that the location of the insertion site is not the abdomen or the buttock of the user of the CGM system. An indication is generated, for display in a user interface of a display device, of an error component included in the glucose data based on the location of the insertion site (block 2010). In some examples, the adaptive system 314 generates the indication of the error component.

Example System and Device

FIG. 21 illustrates an example system generally at 2100 that includes an example computing device 2102 that is representative of one or more computing systems and/or devices that may implement the various techniques described herein. This is illustrated through inclusion of the CGM platform 112. The computing device 2102 may be, for example, a server of a service provider, a device associated with a client (e.g., a client device), an on-chip system, and/or any other suitable computing device or computing system.

The example computing device 2102 as illustrated includes a processing system 2104, one or more computer-readable media 2106, and one or more I/O interfaces 2108 that are communicatively coupled, one to another. Although not shown, the computing device 2102 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 2104 is representative of functionality to perform one or more operations using hardware. Accordingly, the processing system 2104 is illustrated as including hardware elements 2110 that may be configured as processors, functional blocks, and so forth. This may include implementation in hardware as an application-specific integrated circuit or other logic device formed using one or more semiconductors. The hardware elements 2110 are not limited by the materials from which they are formed or the processing mechanisms employed therein. For example, processors may comprise semiconductor(s) and/or transistors (e.g., electronic integrated circuits (ICs)). In such a context, processor-executable instructions may be electronically-executable instructions.

The computer-readable media 2106 is illustrated as including memory/storage 2112. The memory/storage 2112 represents memory/storage capacity associated with one or more computer-readable media. The memory/storage component 2112 may include volatile media (such as random access memory (RAM)) and/or nonvolatile media (such as read only memory (ROM), Flash memory, optical disks, magnetic disks, and so forth). The memory/storage component 2112 may include fixed media (e.g., RAM, ROM, a fixed hard drive, combinations thereof, and so forth) as well as removable media (e.g., Flash memory, a removable hard drive, an optical disc, combinations thereof, and so forth). The computer-readable media 2106 may be configured in a variety of other manners, as described in further detail below.

Input/output interface(s) 2108 are representative of functionality to enable a user to enter commands and/or information to computing device 2102, and to enable information to be presented to the user and/or other components or devices using various input/output devices. Examples of input devices include a keyboard, a cursor control device (e.g., a mouse), a microphone, a scanner, touch functionality (e.g., capacitive or other sensors configured to detect physical touch), a camera (e.g., a device configured to employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 2102 may be configured in a variety of ways as further described below to support user interaction.

Various techniques may be described herein in the general context of software, hardware elements, or program modules. Generally, program modules include routines, programs, objects, elements, components, data structures, and so forth that perform particular tasks or implement particular abstract data types. The terms “module,” “functionality,” and “component” as used herein generally represent software, firmware, hardware, or combinations thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 2102. By way of example, and not limitation, computer-readable media may include “computer-readable storage media” and “computer-readable signal media.”

“Computer-readable storage media” may refer to media and/or devices that enable persistent and/or non-transitory storage of information, in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 2102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 2110 and computer-readable media 2106 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described herein.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 2110. The computing device 2102 may be configured to implement particular instructions and/or functions corresponding to the software and/or hardware modules. Accordingly, implementation of a module that is executable by the computing device 2102 as software may be achieved at least partially in hardware, e.g., through use of computer-readable storage media and/or hardware elements 2110 of the processing system 2104. The instructions and/or functions may be executable/operable by one or more articles of manufacture (for example, one or more computing devices 2102 and/or processing systems 2104) to implement techniques, modules, and examples described herein.

The techniques described herein may be supported by various configurations of the computing device 2102 and are not limited to the specific examples of the techniques described herein. This functionality may also be implemented all or in part through use of a distributed system, such as over a “cloud” 2114 via a platform 2116 as described below.

The cloud 2114 includes and/or is representative of a platform 2116 for resources 2118. The platform 2116 abstracts underlying functionality of hardware (e.g., servers) and software resources of the cloud 2114. The resources 2118 may include applications and/or data that can be utilized while computer processing is executed on servers that are remote from the computing device 2102. Resources 2118 can also include services provided over the Internet and/or through a subscriber network, such as a cellular or Wi-Fi network.

The platform 2116 may abstract resources and functions to connect the computing device 2102 with other computing devices. The platform 2116 may also serve to abstract scaling of resources to provide a corresponding level of scale to encountered demand for the resources 2118 that are implemented via the platform 2116. Accordingly, in an interconnected device embodiment, implementation of functionality described herein may be distributed throughout the system 2100. For example, the functionality may be implemented in part on the computing device 2102 as well as via the platform 2116 that abstracts the functionality of the cloud 2114.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

1-13. (canceled)

14. A method implemented by a computing device, the method comprising:
receiving glucose data describing user glucose values measured by a glucose sensor of a continuous glucose monitoring (CGM) system;
accessing non-glucose data describing historic heart rate variability values of a user of the CGM system;
determining a modification amount based on the non-glucose data;
generating modified glucose data by modifying the user glucose values based on the modification amount; and
generating an indication of the modified glucose data for display in a user interface of a display device.

15. The method as described in claim 14, further comprising:
identifying an error component included in the glucose data based on the historic heart rate variability values of the user; and
determining the modification amount based on the error component.

16. The method as described in claim 15, wherein the modified glucose data does not include the error component.

17. The method as described in claim 15, further comprising:
determining a risk classification for the error component; and
generating an indication of the risk classification for display in the user interface of the display device.

18. The method as described in claim 14, wherein historic heart rate variability values are measured by a heart rate monitor of the CGM system.

19. A method implemented by a computing device, the method comprising:
receiving session data describing historic user glucose values measured by a glucose sensor of a continuous glucose monitoring (CGM) system;
generating modified session data by removing historic user glucose values from the session data that were measured by the glucose sensor during a temporal window that begins at a time corresponding to a timestamp of an oldest historic user glucose value described by the session data;
generating a glucose value report based on the modified session data; and
generating an indication of the glucose value report for display in a user interface of a display device.

20. The method as described in claim 19, wherein the session data is received from a virtual container that limits access to the session data based on a risk classification associated with the access to the session data.

21. The method as described in claim 19, wherein the temporal window ends at time that is 24 hours after the time corresponding to the timestamp.

22. A method implemented by a computing device, the method comprising:
receiving glucose data describing user glucose values measured by a glucose sensor of a continuous glucose monitoring (CGM) system;
accessing non-glucose data describing historic perspiration values of a user of the CGM system;
determining a modification amount based on the non-glucose data;
generating modified glucose data by modifying the user glucose values based on the modification amount; and
generating an indication of the modified glucose data for display in a user interface of a display device.

23. The method as described in claim 22, further comprising:
identifying an error component included in the glucose data based on the historic perspiration values of the user; and
determining the modification amount based on the error component.

24. The method as described in claim 23, wherein the modified glucose data does not include the error component.

25. The method as described in claim 23, further comprising:
determining a risk classification for the error component; and
generating an indication of the risk classification for display in the user interface of the display device.

26. A method implemented by a computing device, the method comprising:
receiving glucose data describing user glucose values measured by a glucose sensor of a continuous glucose monitoring (CGM) system;
accessing non-glucose data describing historic steps taken by a user of the CGM system;
predicting a glucose value event for the user glucose values based on the historic steps taken by the user of the CGM system;
determining that the glucose value event did not occur based on the glucose data;
generating modified glucose data by modifying the user glucose values because the glucose value event did not occur; and
generating an indication of the modified glucose data for display in a user interface of a display device.

27. The method as described in claim 26, wherein the non-glucose data is generated at least partially from forces measured by an accelerometer of the CGM system.

28. The method as described in claim 26, wherein the glucose data includes an error component because the glucose value event did not occur and wherein the modified glucose data does not include the error component.

29. The method as described in claim 26, further comprising generating a confirmation prompt for display in the user interface of the display device to receive a confirmation indication from a user of the CGM system, the confirmation indication confirming the glucose value event did not occur.

30-33. (canceled)
